(19) United States
(12) Patent Application Publication
(10) Pub. No.: US 2022/0040255 A1
(43) Pub. Date: Feb. 10, 2022

(54) CHEMOTHERAPEUTIC REMODELING OF THE GUT MICROBIOME

(71) Applicant: THE SCRIPPS RESEARCH INSTITUTE, La Jolla, CA (US)

(72) Inventors: Reza M. Ghadiri, San Diego, CA (US); Luke J. Leman, Encinitas, CA (US); Poshen B. Chen, San Diego, CA (US); Yannan Zhao, La Jolla, CA (US); Ali Torkamani, San Diego, CA (US); Audrey Black, San Diego, CA (US)

(21) Appl. No.: 17/280,660

(22) PCT Filed: Sep. 26, 2019
(86) PCT No.: PCT/US19/53155

§ 371 Date: Mar. 26, 2021

Related U.S. Application Data
(60) Provisional application No. 62/737,547, filed on Sep. 27, 2018.

(51) Int. Cl. A61K 38/12; C12Q 1/02; C12Q 1/689; A61P 1/00

(52) U.S. Cl. CPC A61K 38/12; C12Q 1/025; C12Q 2600/136; A61P 1/00; C12Q 1/689

(57) ABSTRACT

The present invention provides methods for remodeling gut microbiome to a desired state. The invention also provides in vitro screening platform for identifying novel agents that can remodel dysfunctional gut microbiome.

### CROSS-REFERENCE TO RELATED APPLICATIONS

The subject patent application claims the benefit of priority to U.S. Provisional Patent Application No. 62/737,547 (filed Sep. 27, 2018). The full disclosure of the priority application is incorporated herein by reference in its entirety and for all purposes.

### STATEMENT OF GOVERNMENT SUPPORT

This invention was made with government support under grant numbers HL104462, GM052190, and CA108304 awarded by the National Institutes of Health and grant numbers N00014-94-1-0365, and N00014-94-1-0362 awarded by the Office of Naval Research. The government has certain rights in the invention.

### BACKGROUND OF THE INVENTION

The microbial community colonizing the mammalian gut plays a fundamental role in the host physiology and health. In humans and animals, dysfunctional gut microbiomes have been associated with the development of several chronic diseases such as cardiovascular disease, obesity, diabetes, and central nervous system disorders. Importantly, consumption of the calorically rich Western diet (WD) can transform the gut microbiome into a dysfunctional state, promoting systemic inflammation and progression of chronic diseases.

Remodeling an existing dysfunctional gut microbiome within a living human or animal, esp., in a targeted fashion, represents valuable tools for treating and preventing disease progression. The present invention is directed to novel means for meeting such unmet needs in the art.

### SUMMARY OF THE INVENTION

In one aspect, the invention provides methods for identifying agents that remodel gut microbiome of a subject. These methods entail (a) obtaining a gut microbiota sample from the subject, (b) inoculating and incubating the gut microbiota sample in a growth media in the presence of a plurality of test compounds, (c) assessing effect of the test compounds on remodeling the microbiota, and (d) identifying from the test compounds one or more compounds that remodel the gut microbiome. In some embodiments, effect of the test compounds on remodeling the microbiota is assessed by determining activity and selectivity of each test compound for remodeling the microbiota. Some methods of the invention are directed to identifying agents that can remodel dysfunctional gut microbiome that is associated with a disease afflicted by the subject.

In some methods, assessing effect of the test compounds on remodeling the microbiota involves detecting an alteration in gut microbiota transcriptome. For example, alteration in gut microbiota transcriptome can be detected en masse by next-generation sequencing of bacterial mRNA transcripts. In some methods, assessing effect of the test compounds on remodeling the microbiota involves determining the ratio of Bacteroidetes to Firmicutes in the microbiome or the relative abundances of gut microbiota taxa. The relative bacterial abundances, which can be used for determining the ratio of Bacteroidetes to Firmicutes, can in turn be determined by sequencing of the 16S rRNA amplicon. In some preferred embodiments, the identified agents remodel the microbiota to a desired alternative state without adversely affecting its diversity. In some methods, the employed test compounds are a library of cyclic peptide having a sequence of from four to about sixteen amino acid residues or analogs thereof, which are alternating D- and L-residues along partial or entire sequence of the peptide.

In another aspect, the invention provides methods for remodeling imbalanced or dysfunctional gut microbiota in a subject. These methods involve administering to the subject a pharmaceutical composition that contains a therapeutically effective amount of a cyclic peptide having a sequence of from four to about sixteen amino acid residues or analogs thereof, which are alternating D- and L-residues along partial or entire sequence of the peptide. In some embodiments, the methods are directed to remodeling microbiome in subjects that are afflicted with or at risk of developing hypercholesterolemia, a cardiovascular disorder, an atherosclerotic vascular disease, a cerebrovascular disease, aneurysm, a peripheral vascular disease or intermittent claudication. Preferably, the employed cyclic peptide remodels the gut microbiota in the subject to a functional state without adversely affecting its diversity. In various embodiments, the pharmaceutical composition can be administered to the subject orally, intravenously, subcutaneously or intraperitoneally.

In some embodiments, the employed cyclic peptide contains alternating D- and L-α-amino acid residues along its entire sequence. In some of these embodiments, the cyclic peptide compound has a sequence formula of c[B-J-U1-X-U2-Z], wherein B is a peptide segment containing at least 2 hydrophobic amino acid residues or analogs thereof; J contains a positively charged amino acid residue, a polar uncharged amino acid residue, or an analog thereof, one or both of U1 and U2 contain a negatively charged amino acid residue, a polar uncharged amino acid residue or analog thereof; X contains a polar uncharged amino acid residue, a His residue or an analog thereof, and Z contains Asn, Gln, a charged amino acid residue, or an analog thereof. In various embodiments, the B motif in the sequence of the employed cyclic peptide can contain 2, 3, 4, 5, 6, or 7 hydrophobic amino acid residues or analogs thereof. In some of these methods, the B motif contains 3 hydrophobic amino acid residues or analogs thereof. For example, the B motif can be ^{D}Trp-Leu-^{D}Trp, ^{D}Tyr-Leu-^{D}Tyr, ^{D}Trp-Trp-^{D}Trp, ^{D}Phe-Leu-^{D}Trp, Trp-^{D}Leu-Trp, Tyr-^{D}Leu-Tyr, Trp-^{D}Trp-Trp, or Phe-^{D}Leu-Trp. In some embodiments, the J residue in the sequence of the employed cyclic peptide can be Lys, Arg, Ser, His, Orn (ornithine), diaminobutyric acid or diaminopropionic acid. In some other embodiments, the J residue can be naphthylalanine (Nal), homoleucine (Hml), or 2-amino-octanoic acid (Aoc). In some embodiments, the U1 and U2 residues in the sequence of the employed cyclic peptide can be each independently a ^{D}Asp, ^{D}Glu or ^{D}Ser residue. In some embodiments, the X residue in the sequence of the employed cyclic peptide can be Asn or Gln. In some embodiments, the Z residue in the sequence of the employed cyclic peptide can be a positively charged residue, e.g., Lys, Arg, His, Orn (ornithine) or diaminobutyric acid.

In some preferred embodiments, the employed cyclic peptide has a B motif of ^{D}Trp-Leu-^{D}Trp or ^{D}Tyr-Leu-^{D}Tyr; a J residue of Lys, Arg, or Ser; U1 and U2 residues each independently being ^{D}Asp, ^{D}Glu or ^{D}Ser; a X residue of Asn or Gln; and a Z residue of Lys, Arg, Orn (ornithine) or diaminobutyric acid. In some embodiments of the invention, the specific cyclic peptide administered to the subject is any one of c[wLwReQeR] (SEQ ID NO:11), c[wLwKhShK] (SEQ ID NO:1), c[wLwKkKr] (SEQ ID NO:17), c[WlWlKhKr] (SEQ ID NO:18), c[wLfKwKkK] (SEQ ID NO:2), c[WlWwKkKk] (SEQ ID NO:20), c[wLhLwKrK] (SEQ ID NO:21), c[WlWlKrFr] (SEQ ID NO:19), c[FwHlYoHq] (SEQ ID NO:12), c[WlLlKkKs] (SEQ ID NO:7), c[wLlWkKkS] (SEQ ID NO:5), c[wWwKsKsK] (SEQ ID NO:8), c[lLwHoK] (SEQ ID NO:24), c[wLyKkK] (SEQ ID NO:22), c[wFkSkSkS] (SEQ ID NO:3), and c[lFlAlKhK] (SEQ ID NO:10), c[WwLlHsKk (SEQ ID NO:4), c[YlYlYkSo] (SEQ ID NO:14), c[fWwYqHhQ] (SEQ ID NO:15), c[fVwYkK] (SEQ ID NO:23), c[LlWhQk] (SEQ ID NO:6), c[WwQoHdKt] (SEQ ID NO:27), c[WlWlWkSk] (SEQ ID NO:9), c[wLeLwKsK] (SEQ ID NO:16), c[wLwSeQhK] (SEQ ID NO:25), c[YlWyKhAe] (SEQ ID NO:13), c[YwElYsKq] (SEQ ID NO:26), c[wLwSeQeO] (SEQ ID NO:28), and c[wLlEeKkN] (SEQ ID NO:29).

A further understanding of the nature and advantages of the present invention may be realized by reference to the remaining portions of the specification and claims.

### DESCRIPTION OF THE DRAWINGS

FIG. 1. Peptide treatment reduced WD-induced inflammation in LDLr^{-/-} mice via several gut microbiota-dependent mechanisms. (a) Expression of tight junction markers Tjpl and Ocln in small intestine of vehicle- or peptide c[wLwReQeR] (SEQ ID NO:11)-treated WD-fed animals, as determined by rtPCR (n=3 per group). (b) Representative images of ileum sections showing the recovery in intestinal villi width after cyclic peptide treatment (scale bars, 300 μm). (c) Quantification of intestinal villi width in the ileum from vehicle or peptide treated animals on CHD or WD (n=6-8 per group). (d) Composition of regulatory T cell population in lamina propria from vehicle or peptide treated animals, as indicated by the ratio of Helios⁺ Treg cells to Th17 cells and RORγt⁺ Treg cells in wild type (WT) or LDLr^{-/-} mice. The graphs are shown as the mean±SD, n=3-5 mice per group. (e) Peptide treatment reduced the levels of certain hepatic cytokines/chemokines, as indicated by luminex analysis (n=4-5 per group). Results for the complete panel of 32 cytokines/chemokines are shown in Table 6.

FIG. 2. Changes in gut microbiota composition and biodiversity due to WD feeding in vivo and peptide treatment in vitro. (a) The alpha diversity (biodiversity) of gut microbiota from cecum of LDLr^{-/-} mice cultured in vitro for 24 h with the indicated cyclic peptides 1-29 (SEQ ID NOs: 1-29) (25 μM). Two broad-spectrum antibiotics, 4 μg/mL ampicillin (Amp) and 8 μg/mL chloramphenicol (Chm), were used as positive controls in this assay. The dashed line indicates the biodiversity in the cultured, untreated control sample. (b) The ratio of bacteroidetes to firmicutes in the in vitro cultured microbiota samples after cyclic peptide treatment. The dashed line indicates the ratio of bacteroidetes to firmicutes in the cultured, untreated control sample.

### DETAILED DESCRIPTION OF THE INVENTION

In humans and mammals, a Western diet (WD) can induce imbalances in gut microbiota, in terms of species composition and function, which strongly correlate with the development of several chronic diseases. The present invention is derived in part from the discoveries by the inventors that self-assembling cyclic D,L-α-peptides selected by using an in vitro en masse screening protocol function as bacterial growth modulators to remodel a Western diet-induced imbalance in the gut microbiome and thus prevent atherosclerosis development in LDLr^{-/-} mice. Specifically, daily oral administration of selected peptides to mice remodeled the gut microbiome and caused diverse biological effects in the host, including marked reductions in plasma total cholesterol levels and atherosclerotic plaques. There was extensive reprogramming of the microbiome transcriptome and host gene expression levels, suppressed production of several pro-inflammatory cytokines, improved gut barrier integrity, increased populations of intestinal Helios positive regulatory T cells (Helios+ Treg) and rebalanced levels of disease-relevant metabolites, such as short-chain fatty acids (SCFAs) and bile acids. The ability to chemically manipulate the gut microbiome in a targeted fashion within a living organism provides not only an additional tool for deciphering the chemical biology of the gut microbiome, but also an avenue for advancing personalized therapeutic agents.

In accordance with these discoveries, the present invention provides screening platforms that can be used to identify novel agents that are effective to remodel the gut microbiome to a desired alternative state, e.g., a functional state as seen in mice fed with a Chow diet (CHD). Also provided in the invention are therapeutic uses or methods for employing the various cyclic D,L-α-peptides described herein to provide beneficial microbiome remodeling effects in various subjects with imbalances in gut microbiota. The following description provides a more detailed guidance for practicing the various methods of the invention.

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1st ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). In addition, the following definitions are provided to assist the reader in the practice of the invention.

The term “agent” includes any substance, molecule, element, compound, entity, or a combination thereof. It includes, but is not limited to, e.g., protein, polypeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” are used interchangeably herein.

Amino acids having both the amine and carboxylic acid groups attached to the first (α-) carbon atom have particular importance in biochemistry. They include the 23 “proteinogenic” (“protein-building”) amino acids which combine into peptide chains to form the building blocks of a vast array of proteins. These are all L-stereoisomers (“left-handed” isomers) although a few D-amino acids (“right-handed”) occur in bacterial envelopes and some antibiotics. 20 of the 23 proteinogenic amino acids are encoded directly by triplet codons in the genetic code and are known as “standard” amino acids. The other three (“non-standard” or “non-canonical”) are pyrrolysine (found in methanogenic organisms and other eukaryotes), selenocysteine (present in many noneukaryotes as well as most eukaryotes), and N-Formylmethionine.

Of the standard α-amino acids, all but glycine can exist in either of two enantiomers, called L or D amino acids, which are mirror images of each other. While L-amino acids represent all of the amino acids found in proteins during translation in the ribosome, D-amino acids are found in some proteins produced by enzyme posttranslational modifications after translation and translocation to the endoplasmic reticulum, as in exotic sea-dwelling organisms such as cone snails. They are also abundant components of the peptidoglycan cell walls of bacteria, and D-serine may act as a neurotransmitter in the brain.

Aside from the 23 proteinogenic amino acids, there are many other amino acids that are called non-proteinogenic or non-standard. Those either are not found in proteins (for example carnitine and gamma-aminobutyric acid), or are not produced directly and in isolation by standard cellular machinery (for example, hydroxyproline and selenomethionine). Non-standard amino acids that are found in proteins are formed by posttranslational modification, which is modification after translation during protein synthesis. Some nonstandard amino acids are not found in proteins. Examples include lanthionine, 2-aminoisobutyric acid, dehydroalanine, and the neurotransmitter gamma-aminobutyric acid (GABA). Non-standard amino acids often occur as intermediates in the metabolic pathways for standard amino acids—for example, ornithine and citrulline occur in the urea cycle, part of amino acid catabolism.

The side-chain of an amino acid can make it a weak acid or a weak base, and a hydrophile if the side-chain is polar or a hydrophobe if it is nonpolar. Amino acids can be classified by the properties of their side-chain into non-polar hydrophobic residues, polar but uncharged hydrophilic residues, and polar charged residues. Among the 20 standard proteinogenic amino acids, non-polar hydrophobic residues include alanine, glycine, valine, leucine, isoleucine, proline, phenylalanine, and methionine. Polar uncharged residues include asparagine, glutamine, serine, threonine, tyrosine, cysteine and tryptophan. Polar and charged amino acids include positively charged basic residues (lysine, arginine and histidine) and negatively charged acidic residues (aspartic acid and glutamic acid).

As used herein, “amino acid residues or analogs thereof” encompass naturally occurring amino acids, including both proteinogenic and non-proteinogenic amino acids. Naturally occurring amino acids are those encoded by the genetic code, as well as those amino acids that are later posttranslationally modified (e.g., hydroxyproline, γ-carboxyglutamate, and O-phosphoserine) or otherwise naturally existing in nature. In addition to natural amino acid residues, amino acid residues or analogs present in the cyclic peptides of the invention also encompass non-naturally existing amino acid analogs or derivatives, e.g., synthetic amino acid derivatives and amino acid mimetics that function in a manner similar to the naturally occurring amino acids. The term “amino acid residues or analogs” specifically encompasses both L-form and D-form of amino acid residues. Amino acid analogs refer to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs can have modified R groups (e.g., norleucine) or modified peptide backbones, but retain the same basic chemical structure as a naturally occurring amino acid. Typically, the cyclic peptides of the invention harbor both L-form and D-form of proteinogenic or non-proteinogenic residues, as well as other amino acid derivative or analogs.

The term “analog” or “derivative” is used herein to refer to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs to identify variants of known compounds having improved traits (such as higher binding affinity for a target molecule) is an approach that is well known in pharmaceutical chemistry.

The Bacteroidetes to Firmicutes ratio refers to the relative proportions of two organisms present in the gut microbiota. Commonly present in the gut microbiota are members of the gram-positive Firmicutes and the gram-negative Bacteroidetes phyla, with several other phyla, including the Actinobacteria, Fusobacteria and Verrucomicrobia, present at subdominant levels. The Bacteroidetes to Firmicutes ratio (B/F ratio) has been extensively examined for human and mouse gut microbiota. It has been shown by multiple studies that the B/F ratio is correlated with obesity and other diseases.

Administration “in conjunction with” one or more other therapeutic agents includes simultaneous (concurrent) and consecutive administration in any order.

The term “contacting” has its normal meaning and refers to combining two or more agents (e.g., polypeptides or small molecule compounds) or combining agents and cells. Contacting can occur in vitro, e.g., combining two or more agents or combining an agent and a cell or a cell lysate in a test tube or other container. Contacting can also occur in a cell or in situ, e.g., contacting two polypeptides in a cell by coexpression in the cell of recombinant polynucleotides encoding the two polypeptides, or in a cell lysate. Contacting can also occur inside the body of a subject, e.g., by administering to the subject an agent which then interacts with the intended target (e.g., a tissue or a cell).

As used herein, microbiota refers to the ecological community of commensal, symbiotic and pathogenic microorganisms found in and on all multicellular organisms, including plants and animals. A microbiota includes bacteria, archaea, protists, fungi and viruses which are predominantly found in the gastrointestinal tract, but also in other exposed tissues, such as the skin, upper respiratory and urogenital tracts. Microbiota have been found to be crucial for immunologic, hormonal and metabolic homeostasis of their host. The synonymous term microbiome describes either the collective genomes of the microorganisms that reside in an environmental niche or the microorganisms themselves. The microbiome and host emerged during evolution as a synergistic unit from epigenetics and genetic characteristics, sometimes collectively referred to as a holobiont.

Functional microbiome state (or functional microbiome) refers to healthy microbiome that has normal functions of the microbiome ecosystem, including the production of important resources, bioconversion of nutrients, and protection against pathogenic microbes. Dysfunctional or imbalanced microbiome state refers to a state of microbiome that has impaired beneficial functions of the microbiome ecosystem. The dysfunction is due to the microorganisms in the gut (including bacteria) not living in mutual accord, disturbed balance between “protective” versus “harmful” intestinal microorganisms, and/or overgrowth of “pathobionts” (i.e., normally good bacteria). The dysfunction or imbalance can be caused by (i) loss of beneficial microbial organisms perhaps through the use of antibiotics, (ii) expansion of pathobionts or potentially harmful microorganisms because of too much processed foods, and/or (iii) loss of overall microbial diversity. A number of human diseases are associated with dysfunctional microbiome, including autoimmune and auto inflammatory disorders, such as allergies, cardio vascular, metabolic disorders (diabetes, obesity and non-alcoholic fatty liver disease), various cancers and inflammatory bowel disease such as Crohn's and ulcerative colitis (UC), celiac disease, and neurological disorders including autism.

Remodeling of the gut microbiome refers to any process that can alter the composition of the bacterial species and/or reprogram its transcriptome in a deliberate fashion. In some embodiments, the gut microbiome to be remodeled is dysfunctional, e.g., associated with a disease or disorder. The remodeling is intended to change the dysfunctional state of the microbiome into a healthy and functional state.

The term “subject” for purposes of treatment refers to any animal classified as a mammal, e.g., human and non-human mammals. Examples of non-human animals include dogs, cats, cattle, horses, sheep, pigs, goats, rabbits, and etc. Unless otherwise noted, the terms “patient” or “subject” are used herein interchangeably. Preferably, the subject is human.

The term “treating” or “alleviating” includes the administration of compounds or agents to a subject to prevent or delay the onset of the symptoms, complications, or biochemical indicia of a disease or condition (e.g., imbalanced gut microbiota), alleviating the symptoms or arresting or inhibiting further development of the disease or condition. Subjects in need of treatment include those already suffering from the disease or disorder. They also encompass ones who are at risk of developing the disorder, e.g., one at risk of developing dysfunctional microbiome. Treatment may be prophylactic (to prevent or delay the onset of the disease, or to prevent the manifestation of clinical or subclinical symptoms thereof) or therapeutic suppression or alleviation of symptoms after the manifestation of the disease. In the treatment of a disease or disorder associated with or mediated by dysfunctional microbiome, a therapeutic agent may directly decrease the pathology of the disease, or render the disease more susceptible to treatment by other therapeutic agents.

A “therapeutically effective amount” is intended for a minimal amount of active agent which is necessary to impart therapeutic benefit to a subject. Thus, a “therapeutically effective amount” administered to a subject is such an amount which induces, ameliorates or otherwise causes an improvement in the pathological symptoms, disease progression or physiological conditions associated with a disorder or resistance to succumbing to a disorder.

Disorders mediated by or associated with dysfunctional gut microbiome refer to any or diseases or conditions that implicate imbalanced gut microbiome as a cause or contributing factor in their development. Examples include, e.g., chronic diseases such as cardiovascular disease, obesity, diabetes, and central nervous system disorders.

In one aspect, the invention provides methods of employing an in vitro model of gut microbiome described herein to identify novel agents that can effectively remodel a gut microbiome to a desired alternative state. In some embodiments as exemplified herein, the screening methods of the invention employ fresh gut microbiome sample obtained from a subject (e.g., a mouse with distinct functional and dysfunctional gut microbiome states), and utilize an en masse screening format to assess effects of candidate compounds on the community of commensal bacteria species present in the microbiome all at once. It was found that this screening format is more data rich, internally consistent, and can account for potential indirect growth-modulating effects that might exist within the bacterial community (vide infra).

Some screening methods of the invention are directed to identifying novel compounds that are capable of remodeling dysfunctional gut microbiome associated with a disease or condition. The dysfunctional gut microbiome can be present in a subject suffering from a number of inflammatory disorders, chronic diseases, metabolic disorders such as obesity, and cardiovascular diseases. Typically, the screening methods require isolation of a gut microbiota sample from a subject afflicted or at risk of developing any of these diseases or conditions. In some embodiments, the gut microbiota sample can be an optimized fecal sample that is collected from the subject. Collection and optimization of fecal samples for microbiome study can be readily performed in accordance with the protocols exemplified herein or well known in the art. See, e.g., Thomas et al., Future Microbiol. 10:1485-1504, 2015; Vandeputte et al. Gut 65:57-62, 2016; Abrahamson et al., Contemporary Clinical Trials Communications 7: 158-162, 2017; and Wu et al., J. Formos. Med. Assoc. 17:30857-4, 2018. The subject can be a human patient suffering from dysfunctional gut microbiota or a non-human animal with dysfunctional gut microbiota. Alternatively, as exemplified herein, the subject can also be a non-human animal model (e.g., mouse model) for a human disorder with manifested dysfunctional gut microbiota. For example, dysfunction gut microbiota sample can be obtained from Western diet (WD) fed LDLr^{-/-} mice as exemplified herein or WD fed wildtype mice as reported in the art, e.g., Carmody et al., Cell Host Microbe 17, 72-84, 2015. After isolating the gut microbiota sample from the subject, the sample is inoculated and incubated in a growth media (e.g., 96-well plates) in the presence of candidate compounds under appropriate experimental condition to allow interaction of the compounds with organisms in the microbiome sample. This is followed by examining effects, if any, of the candidate compounds on remodeling the microbiome. Positive agents are identified from the candidate compounds if they are able to remodel the microbiota sample to a desired alternative state, e.g., similar to gut microbiome of a healthy human subject or a functional microbiome state as in CHD fed mice. Preferably, the identified compound remodels the microbiota sample to a desired alternative state without adversely affecting its diversity. Remodeling effects of candidate compounds can be determined via any of the various assays described below.

In the screening methods of the invention, candidate agents can be screened for one or more activities that evidence their ability to remodel gut microbiome to a desired alternate state. In some embodiments, effect of the candidate compounds on remodeling the microbiota is determined by examining the microbiota transcriptome of bacteria from a plurality of microbiota samples (e.g., from WD fed mice) that have been treated with different candidate compounds. The compound treated microbiota transcriptome can be compared to that of negative control microbiota (e.g., from untreated WD fed mice) and/or that of positive control (e.g., functional gut microbiota from CHD fed mice). Examination and comparison of the microbiota transcriptome can be readily performed by standard protocols or techniques well known and routinely practiced in the art. These include, e.g., next-generation sequencing of bacterial mRNA transcripts as exemplified herein. In some other embodiments, remodeling effect of a given candidate compound can be correlated with an altered composition of microorganism species in the microbiome sample. For example, effect of the test compounds on remodeling the microbiota can be monitored by determining the ratio of Bacteroidetes to Firmicutes. The ratio of Bacteroidetes to Firmicutes in the microbiome sample can be determined by, e.g., sequencing of 16S rRNA amplicon as exemplified herein. In some preferred embodiments, the identified compound remodels the microbiota sample to a desired alternative state without adversely affecting its diversity.

In some other embodiments, the candidate compounds can be examined for ability to reduce plasma total cholesterol levels and atherosclerotic plaques. In still some other embodiments, the candidate compounds can be assessed for ability to suppress production of several pro-inflammatory cytokines. In some embodiments, the candidate compounds are monitored for ability to improve gut barrier integrity. In some other embodiments, the candidate compounds can be examined for activity in increasing populations of intestinal Helios positive regulatory T cells (Helios+ Treg). In still some other embodiments, the candidate compounds can be screened for activity in achieving rebalanced levels of disease-relevant metabolites, such as short-chain fatty acids (SCFAs) and bile acids. These assays can all be readily performed in accordance with experimental procedures well known in the art or the specific protocols exemplified herein.

Candidate compounds suitable for the screening methods of the invention can be of any chemical nature. They can include any substance, molecule, element, compound, entity, or a combination thereof. Unless otherwise specified, the terms “agent”, “substance”, and “compound” are used interchangeably herein. In various embodiments, candidate agents or compounds that can be screened with methods of the present invention include polypeptides, beta-turn mimetics, polysaccharides, phospholipids, hormones, prostaglandins, steroids, aromatic compounds, heterocyclic compounds, benzodiazepines, oligomeric N-substituted glycines, oligocarbamates, polypeptides, saccharides, fatty acids, steroids, purines, pyrimidines, derivatives, structural analogs or combinations thereof. Some candidate agents are synthetic molecules, and others natural molecules. Candidate agents can be obtained from a wide variety of sources including libraries of synthetic or natural compounds.

In some preferred embodiments, the candidate agents are small molecule organic compounds, e.g., chemical compounds with a molecular weight of not more than about 1,000 or 500. Preferably, high throughput assays can be adapted and used to screen such small molecules in accordance with methods routinely practiced in the art.

In some embodiments, the candidate agents are naturally occurring proteins or their fragments. Such candidate agents can be obtained from a natural source, e.g., a cell or tissue lysate. Libraries of polypeptide agents can also be prepared, e.g., from a cDNA library commercially available or generated with routine methods. The candidate agents can also be peptides, e.g., peptides of from about 5 to about 30 amino acids, with from about 5 to about 20 amino acids being preferred, and from about 7 to about 15 being particularly preferred. The peptides can be digests of naturally occurring proteins, random peptides, or “biased” random peptides. In some methods, the candidate agents are polypeptides or proteins. The candidate agents can also be nucleic acids. Nucleic acid candidate agents can be naturally occurring nucleic acids, random nucleic acids, or “biased” random nucleic acids. For example, digests of prokaryotic or eukaryotic genomes can be similarly used as described above for proteins.

In some other embodiments, the candidate agents are a library of cyclic peptide having a sequence of from four to about sixteen amino acid residues or analogs thereof, which are alternating D- and L-residues along partial or entire sequence of the peptide. In some embodiments, the candidate agents can be analog compounds or derivatives that can be generated from one or more of the cyclic peptides exemplified herein.

To identify microbiome-remodeling cyclic peptides that have little or no undesired toxicity for mammalian cells, individual cyclic peptides, or libraries of cyclic peptides can be made, followed by screening for microbiota-remodeling activity and toxicity using assays and techniques as exemplified herein and/or that are well known in the art. For example, libraries of peptides can be made using a one-bead-one-compound strategy provided by Lam et al. (97 Chem. Rev. 411-448 (1997) or synthesized on microbeads by a split and pool method of Furka, et al. (37 Int. J. Pept. Prot. Res. 487-493 (1991)). Mass spectrometric sequence analysis techniques enable rapid identification of every peptide within a given library. See, Biemann, K. 193 Methods Enzymol. 455 (1990). In general, synthetic operations, including peptide cyclization, are performed on solid support to avoid laborious and difficult to automate solution-phase operations. Moreover, the final product of the synthesis regimen is generally sufficiently pure for biological assays without laborious purification procedures. Peptide yields from each synthesis can be sufficient for performing 50 to 100 assays. Rapid, automatic mass-spectrometry-based peptide sequence analysis can be performed to identify peptide sequences that have high activity and to discard peptide sequences with low activity.

In some embodiments, the candidate agents to be screened are a combinatorial library of compounds. Combinatorial libraries can be produced for many types of compound that can be synthesized in a step-by-step fashion. Large combinatorial libraries of compounds can be constructed by the encoded synthetic libraries (ESL) method described in WO 95/12608, WO 93/06121, WO 94/08051, WO 95/35503 and WO 95/30642. Peptide libraries can also be generated by phage display methods (see, e.g., WO 91/18980). Libraries of natural compounds in the form of bacterial, fungal, plant and animal extracts can be obtained from commercial sources or collected in the field. Known pharmacological agents can be subject to directed or random chemical modifications, such as acylation, alkylation, esterification, amidification to produce structural analogs.

Combinatorial libraries of peptides or other compounds can be fully randomized, with no sequence preferences or constants at any position. Alternatively, the library can be biased, i.e., some positions within the sequence are either held constant, or are selected from a limited number of possibilities. For example, in some cases, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, or to purines.

In another aspect, the invention provides therapeutic methods of using cyclic peptides to remodel dysfunction gut microbiome. Typically, the cyclic peptides employed in the invention are self-assembling D,L-α-peptides and comprise a sequence of from four to about sixteen amino acid residues or analogs thereof (including pharmaceutically acceptable salt derivatives). Typically, the amino acid residues or analogs thereof present in the cyclic peptides are alternating D- and L-residues along partial or entire sequence of the peptide. In some embodiments, the cyclic peptides have from about six to about ten or twelve alternating D- and L-α-amino acids. In other embodiments, the cyclic peptides having about six or eight alternating D- and L-α-amino acids are employed. Preferably, amino acid residues of the cyclic peptide are alternating D- and L-residues as present in the entire sequence of the peptide. Examples of such peptides are described herein in the Examples below. Additional cyclic peptides that may be used in the invention are described, e.g., in PCT publications WO 95/10535, WO 03/092631, WO 03/092632 and WO 14/165563. Preferably, the cyclic peptides employed in the invention have minimum or no undesired toxicity against normal mammalian cells as noted above, which can be determined, e.g., by hemolysis of erythrocytes.

Cyclic peptides with gut microbiome remodeling activities can be examined by the in vivo and in vitro assay formats described herein. In some embodiments, the employed cyclic peptides can be selected based on their activities to remodel gut microbiota in vivo, e.g., by inducing metabolic changes and transcriptional reprogramming of the gut microbiota in WD fed mice as exemplified herein. In some embodiments, the employed peptides are those that are shown to be able to alter microbiota composition in vitro, e.g., cause species level changes in bacterial abundance as deterred by transcriptome analysis. In some embodiments, peptides with ability to remodel the microbiota community most completely from the dysfunctional state toward the functional state are preferably employed in the practice of the therapeutic methods of the invention. In some embodiments, the employed peptides can reduce the levels of specific bacterial species (lowest summed differences in relative abundance) that were increased in dysfunctional state (e.g., as induced in mice by WD feeding) to levels that are similar to their levels in a functional state of gut microbiome (e.g., in CHD-fed mice).

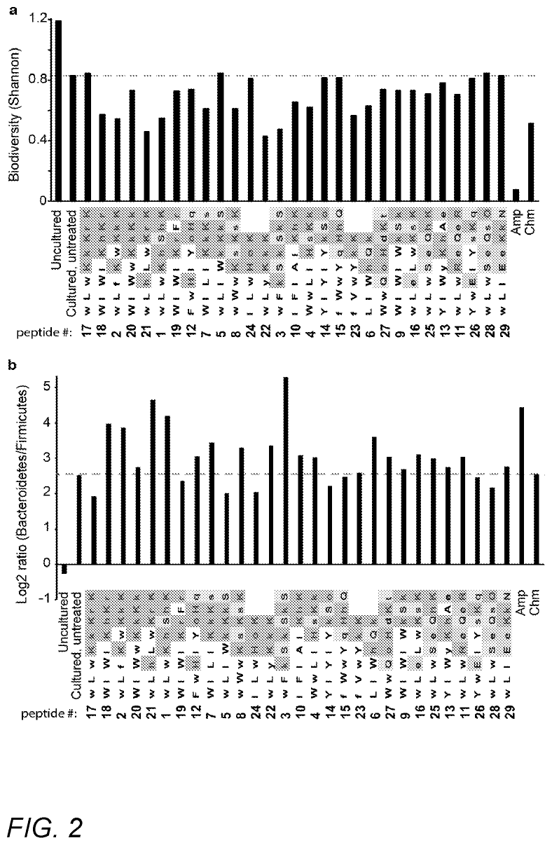

In some preferred embodiments, the cyclic peptides employed in the therapeutic methods of the invention are cyclic D,L-α-peptide sequences active against pathogenic aerobic bacteria. In general, the cyclic D,L-α-peptides do not possess mammalian cell toxicity, and also do not diminish microbiota alpha diversity (the number and evenness of bacterial taxa within the community) as exemplified in FIG. 2a. Preferably, the peptides should not possess broad-spectrum antibacterial activity (e.g., inhibit >95% of growth of ≥25% of species in the community). In some preferred embodiments, the peptides should significantly alter the Bacteroidetes/Firmicutes ratio as determined via in vitro culture.

In some preferred embodiments, cyclic peptides employed in the methods of the invention fall under a sequence formula of c[B-J-U1-X-U2-Z]. In this sequence formula, B is a hydrophobic peptide segment containing at least 2 hydrophobic amino acid residues or analogs thereof. J is moiety containing a positively charged amino acid residue, a polar uncharged amino acid residue, or an analog thereof. In addition, one or both of U1 and U2 contains a negatively charged amino acid residue, a polar uncharged amino acid residue or analog thereof. Flanked by U1 and U2, X moiety or segment represents the center of a polar face or segment of the cyclic peptide and typically contains a polar uncharged amino acid residue, a His residue or an analog thereof. Finally, at the end of the sequence formula of the cyclic peptide, Z moiety or segment contains Asn, Gln, a charged amino acid residue, or an analog thereof.

In some embodiments, the hydrophobic peptide segment B consists of 2, 3, 4, 5, 6 or 7 hydrophobic amino acid residues or analogs thereof. In some preferred embodiments, hydrophobic peptide segment consists of 3 hydrophobic amino acid residues or analogs thereof. Specific examples of the cyclic peptides of the invention are shown in FIG. 2, e.g., Peptide 1—c[wLwKhShK] (SEQ ID NO:1) and Peptide 11—c[wLwReQeR] (SEQ ID NO:11). Other peptides exemplified herein include c[wLwKkKr] (SEQ ID NO:17), c[WlWlKhKr] (SEQ ID NO:18), c[wLfKwKkK] (SEQ ID NO:2), c[WlWwKkKk] (SEQ ID NO:20), c[wLhLwKrK] (SEQ ID NO:21), c[WlWlKrFr] (SEQ ID NO:19), c[FwHlYoHq] (SEQ ID NO:12), c[WlLlKkKs] (SEQ ID NO:7), c[wLlWkKkS] (SEQ ID NO:5), c[wWwKsKsK] (SEQ ID NO:8), c[lLwHoK] (SEQ ID NO:24), c[wLyKkK] (SEQ ID NO:22), c[wFkSkSkS] (SEQ ID NO:3), and c[lFlAlKhK] (SEQ ID NO:10), c[WwLlHsKk (SEQ ID NO:4), c[YlYlYkSo] (SEQ ID NO:14), c[fWwYqHhQ] (SEQ ID NO:15), c[fVwYkK] (SEQ ID NO:23), c[LlWhQk] (SEQ ID NO:6), c[WwQoHdKt] (SEQ ID NO:27), c[WlWlWkSk] (SEQ ID NO:9), c[wLeLwKsK] (SEQ ID NO:16), c[wLwSeQhK] (SEQ ID NO:25), c[YlWyKhAe] (SEQ ID NO:13), c[YwElYsKq] (SEQ ID NO:26), c[wLwSeQeO] (SEQ ID NO:28), and c[wLlEeKkN] (SEQ ID NO:29).

In addition to these specific peptides shown in FIG. 2, derivative peptides which have conservative amino acid substitutions relative to the sequence of the exemplified peptides are also encompassed by the invention. In some embodiments, the cyclic peptides described herein (e.g., compounds shown in SEQ ID NOs:1-29) can be modified via backbone alkylation to identify derivative or analog compounds with similar or improved properties. In some embodiments, salts of carboxyl groups of a peptide or peptide variant of the invention may be prepared in the usual manner by contacting the peptide with one or more equivalents of a desired base such as, for example, a metallic hydroxide base, e.g., sodium hydroxide; a metal carbonate or bicarbonate base such as, for example, sodium carbonate or sodium bicarbonate; or an amine base such as, for example, triethylamine, triethanolamine, and the like.

Some cyclic peptides suitable for use in the therapeutic applications of the invention contain a hydrophobic peptide segment ^{D}Trp-Leu-^{D}Trp, ^{D}Tyr-Leu-^{D}Tyr, ^{D}Trp-^{D}Trp-^{D}Trp, or ^{D}Phe-Leu-^{D}Trp. In some other peptides, the hydrophobic segment contains an enantiomeric tripeptide, e.g., Trp-^{D}Leu-Trp, Tyr-^{D}Leu-Tyr, Trp-^{D}Trp-Trp, or Phe-^{D}Leu-Trp. In some cyclic peptides of the invention, the peptide moiety or segment J, which is adjacent to the hydrophobic segment B, is a single serine residue or a single positively charged residue such as Lys, Arg, His, Orn (ornithine), diaminobutyric acid or diaminopropionic acid. In some other embodiments, J is an amino acid analog or derivative such as naphthylalanine (Nal), homoleucine (Hml), or 2-amino-octanoic acid (Aoc). In some preferred cyclic peptides of the invention, U1 and U2 are each independently a ^{D}Asp, ^{D}Glu or ^{D}Ser residue. In some embodiments, these two acidic or neutral Ser residues are separated by an Asn or Gln residue (X) located in the center of a polar segment of the cyclic peptide. In some preferred embodiments, a positively charged natural or unnatural residue (Z) is present at the end of the sequence of the cyclic peptides, e.g., Lys, Arg, His, Orn (ornithine) or diaminobutyric acid.

Some preferred cyclic peptide compounds of the invention are comprised of about 8 alternating D- and L-form of amino acid residues or (unnatural) amino acid analogs. These peptides have a sequence that falls under the formula c[wLw-J-u1-X-u2-Z]. In this sequence formula, “wLw” denotes a tripeptide segment consisting of^{D}Trp-Leu-^{D}Trp; J is a positively charged amino acid residue or amino acid analog, or serine residue; u1 and u2 are each independently a ^{D}Asp, ^{D}Glu, ^{D}His or ^{D}serine residue; X is Ser residue, Gln residue or Asn residue; and Z is Lys, Arg, His, Orn (ornithine) or diaminobutyric acid.

The gut microbiome remodeling cyclic peptides suitable for the invention may be produced by any conventional automated or manual peptide synthesis methods. General principles and techniques for automated peptide synthesis are well known to those of skill in the art. In some embodiments, the isolated, purified peptides or variants of the invention can be synthesized in vitro, e.g., by the solid phase peptide synthetic method or by enzyme catalyzed peptide synthesis or with the aid of recombinant DNA technology. Solid phase peptide synthetic method is an established and widely used method, which is described in references such as the following: Stewart et al., Solid Phase Peptide Synthesis, W. H. Freeman Co., San Francisco (1969); Merrifield, J. Am. Chem. Soc. 85 2149 (1963); Meienhofer in “Hormonal Proteins and Peptides,” ed.; C. H. Li, Vol. 2 (Academic Press, 1973), pp. 48-267; and Bavaay and Merrifield, “The Peptides,” eds. E. Gross and F. Meienhofer, Vol. 2 (Academic Press, 1980) pp. 3-285. Various automatic synthesizers are commercially available and can be used in accordance with known protocols. The synthesized peptides can be further purified by fractionation on immunoaffinity or ion-exchange columns; ethanol precipitation; reverse phase HPLC; chromatography on silica or on an anion-exchange resin such as DEAE; chromatofocusing; SDS-PAGE; ammonium sulfate precipitation; gel filtration using, for example, Sephadex G-75; ligand affinity chromatography; or crystallization or precipitation from non-polar solvent or nonpolar/polar solvent mixtures. Purification by crystallization or precipitation is preferred.

To remodel a dysfunctional gut microbiome in a subject, the cyclic peptides described herein may be administered in the form of a pharmaceutical composition. As detailed below, a pharmaceutical composition contains a therapeutically effective amount of a microbiome-remodeling cyclic peptide (e.g., Peptide 1 or 11) along with a pharmaceutically acceptable carrier, diluent or excipient in unit dosage form. The invention accordingly also provides pharmaceutical compositions comprising one or more of the microbiome-remodeling cyclic peptides disclosed herein. The invention further provides a use of these cyclic peptides in the preparation of pharmaceutical compositions or medicaments for remodeling gut microbiome in subjects with dysfunctional gut microbiome.

Pharmaceutically acceptable carriers are agents which are not biologically or otherwise undesirable. These agents can be administered to a subject along with a microbiome-remodeling cyclic peptide without causing any undesirable biological effects or interacting in a deleterious manner with any of the components of the pharmaceutical composition. The compositions can additionally contain other therapeutic agents that are suitable for remodeling gut microbiome (e.g., oxidents). Pharmaceutically carriers enhance or stabilize the composition or facilitate preparation of the composition. Pharmaceutically acceptable carriers include solvents, dispersion media, coatings, antibacterial and antifungal agents, isotonic and absorption delaying agents, and the like that are physiologically compatible. The pharmaceutically acceptable carrier employed should be suitable for various routes of administration described herein. Additional guidance for selecting appropriate pharmaceutically acceptable carriers is provided in the art, e.g., Remington: The Science and Practice of Pharmacy, Mack Publishing Co., 20^{th} ed., 2000.

A pharmaceutical composition containing a microbiome-remodeling cyclic peptide described herein and/or other therapeutic agents can be administered by a variety of methods known in the art, e.g., oral administration as exemplified in the Examples below. The routes and/or modes of administration vary depending upon the desired results. Depending on the route of administration, the active therapeutic agent may be coated in a material to protect the compound from the action of acids and other natural conditions that may inactivate the agent. Conventional pharmaceutical practice may be employed to provide suitable formulations to administer such compositions to subjects. Any appropriate route of administration may be employed. These include, but are not limited to, oral, intravenous, parenteral, transcutaneous, subcutaneous, intraperitoneal, intramuscular, intracranial, intraorbital, intraventricular, intracapsular, and intraspinal administration. Depending on the specific conditions of the subject to be treated, either systemic or localized delivery of the therapeutic agents may be used in the treatment.

Pharmaceutical compositions of the invention can be prepared in accordance with methods well known and routinely practiced in the art. See, e.g., Remington: The Science and Practice of Pharmacy, Mack Publishing Co., 20^{th} ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for molecules of the invention include ethylene-vinyl acetate copolymer particles, osmotic pumps, implantable infusion systems, and liposomes. Formulations for inhalation may contain excipients, for example, lactose, or may be aqueous solutions containing, e.g., polyoxyethylene-9-lauryl ether, glycocholate and deoxycholate, or may be oily solutions for administration in the form of nasal drops, or as a gel.

The cyclic peptides for use in the methods of the invention should be administered to a subject in an amount that is sufficient to achieve the desired therapeutic effect (e.g., achieving a healthy and functional microbiome state) in a subject in need thereof. Typically, a therapeutically effective dose or efficacious dose of the cyclic peptide is employed in the pharmaceutical compositions of the invention. Actual dosage levels of the active ingredients in the pharmaceutical compositions of the present invention can be varied so as to obtain an amount of the active ingredient which is effective to achieve the desired therapeutic response for a particular subject, composition, and mode of administration, without being toxic to the subject. The selected dosage level depends upon a variety of pharmacokinetic factors including the activity of the particular compositions of the present invention employed, the route of administration, the time of administration, and the rate of excretion of the particular compound being employed. It also depends on the duration of the treatment, other drugs, compounds and/or materials used in combination with the particular composition employed, the age, gender, weight, condition, general health and prior medical history of the subject being treated, and like factors. Methods for determining optimal dosages are described in the art, e.g., Remington: The Science and Practice of Pharmacy, Mack Publishing Co., 20^{th} ed., 2000. Typically, a pharmaceutically effective dosage would be between about 0.001 and 100 mg/kg body weight of the subject to be treated.

In some embodiments, the present invention provides a packaged pharmaceutical composition for remodeling dysfunctional gut microbiome such as a kit or other container. The kit or container holds a therapeutically effective amount of a pharmaceutical composition for remodeling gut microbiome and optionally also an instruction sheet detailing how to use the pharmaceutical composition. In addition to the pharmaceutical composition containing the microbiome-remodeling cyclic peptide and optional carriers described herein, the kit can additionally contain other reagents for administering the composition.

### EXAMPLES

The following examples are provided to further illustrate the invention but not to limit its scope. Other variants of the invention will be readily apparent to one of ordinary skill in the art and are encompassed by the appended claims.

### Example 1 Cyclic D,L-α-Peptides as Synthetic Growth Modulators of Commensal Bacteria

We examined the utility of self-assembling cyclic D,L-α-peptides—a manmade class of membrane-active supramolecular antimicrobial agents—for use in the directed chemotherapeutic remodeling of a dysfunctional gut microbiome into a functional state. Cyclic peptides with an even number of alternating D-, and L-α-amino acid configuration (typically 6 or 8 residues) can self-assemble under conditions that favor hydrogen bonding (such as in a lipid membrane environment) into extended β-sheet-like hollow tubular structures. By appropriate choice of the sequence, cyclic D,L-α-peptides can selectively partition into bacterial membranes, interrupt transmembrane potential and/or ion gradients, and impair cell growth. Accordingly, we envisioned that the remodeling of a dysfunctional gut microbiome could involve oral administration of a given cyclic D,L-α-peptide sequence that was selected beforehand to possess differential bacterial growth modulating activities against certain members of the gut microbiota. By retarding the growth of certain bacteria in the gut, other unaffected species arise to shift the overall composition of the microbiome over time toward an alternate and potentially beneficial state (vide infra).

### Example 2 Atherosclerosis Model of Functional Vs. Dysfunctional Gut Microbiome

Mice are commonly used for the study of the gut microbiome as they bear similarities to humans in terms of microbiome composition, gastrointestinal phylogenetics, and metabolism. Low-density lipoprotein receptor null (LDLr^{-/-}) mice have a human-like lipoprotein profile and have been widely used as an animal model for diet-induced atherosclerosis. LDLr^{-/-} mice develop hypercholesterolemia and arterial plaques after several weeks on a WD, but not on a Chow diet (CHD). Importantly, feeding a WD to LDLr^{-/-} mice for two weeks caused a widespread remodeling of their gut microbiome as compared to mice fed a CHD (Table 1), consistent with the observed impacts of a WD on the gut microbiome of wild-type mice (Carmody et al., Cell Host Microbe 17, 72-84, 2015). Thus, depending on the diet, LDLr^{-/-} mice exhibit two distinct gut microbiome states (functional and dysfunctional) that are associated with different phenotypic outcomes. We hypothesized that if the WD-induced gut microbiome is causal in the onset and progression of atherosclerosis in LDLr^{-/-} mice, then remodeling the microbiota in vivo (even under the influence of WD) toward a functional CHD state should ameliorate disease progression, thereby testing the therapeutic potential of gut microbiome remodeling.

TABLE 1
List of bacterial taxa in cecurn samples of LDLr^{-/-}
mice for which abundance significantly
changed between CHD feeding and WD feeding.

| | Fold change |
|---|---|
| taxon | (WD/CHD) |
| g_Anaeroplasma | 0.003 |
| f_Elysipelotrichaceae, g_Incertae_Sedis | 0.012 |
| p_Firmicutes, c_unclassified | 0.04 |
| o_RF9, f_unclassified | 0.05 |
| g_Anaerovorax | 0.06 |
| g_Dorea | 0.08 |
| f_Elysipelotrichaceae, g_unclassified | 0.13 |
| g_Rikenella | 0.13 |
| f_Rikenellaceae, g_unclassified | 0.14 |
| g_Papillibacter | 0.14 |
| c_Clostridia, o_unclassified | 0.17 |
| g_Flavonifractor | 0.18 |
| f_vadinBB60, g_unclassified | 0.19 |
| f_Family_XIII, g_unclassified | 0.19 |
| g_Anaerofustis | 0.19 |
| f_Ruminococcaceae, g_Incertae_Sedis | 0.21 |
| g_Oscillospira | 0.23 |
| g_Roseburia | 0.23 |
| g_Gordonibacter | 0.24 |
| p_Proteobacteria, c_unclassified | 0.24 |
| g_Streptococcus | 0.26 |
| g_Odoribacter | 0.27 |
| g_Intestinimonas | 0.29 |
| g_Candidatus_Saccharimonas | 0.31 |
| g_Enterorhabdus | 0.31 |
| g_Anaerotruncus | 0.32 |
| f_Defluviitaleaceae,g_Incertae_Sedis | 0.32 |
| o_Lactobacillales, f_unclassified | 0.32 |
| c_Betaproteobacteria, o_unclassified | 0.34 |
| g_Coprococcus | 0.35 |
| f_Ruminococcaceae, g_unclassified | 0.38 |
| f_Coriobacteriaceae, g_unclassified | 0.39 |
| g_RC9_gut_group | 0.42 |
| g_Oscillibacter | 0.44 |
| o_Clostridiales, f_unclassified | 0.48 |
| g_Acetatifactor | 0.50 |
| g_Lactobacillus | 0.51 |
| f_Desulfovibrionaceae, g_unclassified | 0.53 |
| f_Lachnospiraceae, g_unclassified | 0.53 |
| f_Family_XIII, g_Incertae_Sedis | 0.59 |
| g_Parasutterella | 1.77 |
| g_Desulfovibrio | 1.79 |
| g_Bacteroides | 2.75 |
| g_Butyricicoccus | 3.51 |
| g_Lactococcus | 3.65 |
| g_Allobaculum | 4.19 |
| g_Parabacteroides | 4.24 |
| p_Bacteroidetes, c_unclassified | 6.00 |
| f_Prevotellaceae, g_unclassified | 7.63 |
| g_Bifidobacterium | 19.18 |
| g_Alloprevotella | 97.03 |
| g_Rhizobium | not obs in CHD |
| g_Candidatus_Arthromitus | not obs in CHD |
| f_Rhizobiaceae, g_unclassified | not obs in CHD |

### Example 3 In Vitro Screening Approach to Select Gut Microbiome Remodeling Peptides

Any approach for identifying chemical compounds with potentially useful gut microbiome remodeling profiles, would in practice require an informative in vitro screening process to select compounds that possess the desired range of differential bacterial growth modulating activities. In principle, this goal might be accomplished by in vitro screening of each compound separately against individual cultured commensal bacteria (MIC-type assays) or against the community of species present in the microbiome all at once (en masse screening). The typical MIC assay is used to screen compounds against one bacterial species at a time, which would entail not only a large number of bacterial screens for each assayed compound, but also subsequent comparative data analyses that would be complicated by the differences in growth media and individual bacterial growth profiles. On the other hand, assessing the effects of each compound against the whole gut microbiome in vitro (en masse) would be a more direct approach, but would entail considerable challenges, such as how the gut microbiome is resourced (or assembled) and maintained in vitro to give a reasonable representation of the in vivo community.

We investigated both methods in the present study, but our data show the en masse approach to be superior, as it is more data rich, internally consistent, and can account for potential indirect growth-modulating effects that might exist within the bacterial community (vide infra). We have established the following in vitro compound screening protocol for en masse gut microbiome remodeling. We obtain fresh gut microbiome content from LDLr^{-/-} mice conditioned for at least two weeks on WD by surgically removing cecum under anaerobic conditions. These samples are then mixed with growth media, distributed in 96-well plates, and incubated overnight in the presence of the test peptides. The activity and selectivity of each peptide for remodeling the gut microbiota community was assessed en masse by 16S rRNA sequencing of each well. A number of challenges had to be addressed in developing this protocol, including inoculum handling, choice of growth media, assay duration, etc. Our current assay culture condition maintains 50 bacterial genera (overnight culture), which is fairly representative of the 67 genera found in the uncultured community (Table 2).

TABLE 2
List of bacterial taxa observed in uncultured cecum samples
of LDLr^{-/-} mice and in the in vitro en masse screening assay.

| | Present | | Present |
|---|---|---|---|
| | within in | | within in |
| | vitro | | vitro |
| taxon | culture? | taxon | culture? |
| g_Candidatus Saccharimonas | − | o_Bacteroidales, f_unclassified | + |
| g_Staphylococcus | − | p_Bacteroidetes, c_unclassified | + |
| g_Lactococcus | − | o_Gastranaerophilales, f_unclassified | + |
| g_Anaerofustis | − | g_Mucispirillum | + |
| f_Family_XIII, g_unclassified | − | g_Lactobacillus | + |
| possible_genus_Sk018 | − | g_Streptococcus | + |
| g_Peptococcus | − | o_Lactobacillales, f_unclassified | + |
| g_Oscillospira | − | o_Lactobacillales, f_Incertae_Sedis | + |
| g_Pseudoflavonifractor | − | o_Lactobacillales, f_unclassified | + |
| g_Sporobacter | − | f_Family_XIII, g_Incertae_Sedis | + |
| f_vadinBB60, g_unclassified | − | g_Acetatifactor | + |
| c_Clostridia, o_unclassified | − | g_Blautia | + |
| g_Thalassospira | − | g_Coprococcus | + |
| g_Bilophila | − | f_Lachnospiraceae, g_Incertae_Sedis | + |
| f_Desulfovibrionaceae, g_unclassified | − | g_Marvinbryantia | + |
| g_Ureaplasma | − | g_Roseburia | + |
| g_Akkermansia | − | f_Lachnospiraceae, g_unclassified | + |
| g_Bifidobacterium | + | g_Dehalobacterium | + |
| g_Enterorhabdus | + | f_Peptostreptococcaceae, g_Incertae_Sedis | + |
| g_Olsenella | + | g_Anaerotruncus | + |
| g_Parvibacter | + | f_Ruminococcaceae, g_Incertae_Sedis | + |
| f_Coriobacteriaceae, g_unclassified | + | g_Intestinimonas | + |
| g_Bacteroides | + | g_Oscillibacter | + |
| g_Odoribacter | + | f_Ruminococcaceae, g_unclassified | + |
| g_Parabacteroides | + | o_Clostridiales, f_unclassified | + |
| g_Alloprevotella | + | g_Allobaculum | + |
| g_Prevotella | + | f_Erysipelotrichaceae, g_unclassified | + |
| f_Prevotellaceae, g_unclassified | + | p_Firmicutes, c_unclassified | + |
| g_Alistipes | + | g_Parasutterella | + |
| g_RC9_gut_group | + | c_Betaproteobacteria, o_unclassified | + |
| g_Rikenella | + | g_Desulfovibrio | + |
| f_Rikenellaceae, g_unclassified | + | g_Helicobacter | + |
| f_S24-7, g_unclassified | + | p_unclassified | + |

Our laboratory maintains a library of approximately 1,500 diverse cyclic D,L-α-peptide sequences (pure and spectroscopically characterized) that possess a range of antimicrobial activity and species selectivity. This library was constructed in the course of screening 200,000 cyclic D,L-α-peptides as part of a previous research program aimed at the discovery of cyclic D,L-α-peptide sequences active against pathogenic aerobic bacteria. For the present study we selected 29 cyclic D,L-α-peptides from this library based on sequence diversity and lack of mammalian cell toxicity. Gratifyingly, we observed a range of microbiota remodeling effects in the en masse screen, largely without diminishing microbiota alpha diversity (the number and evenness of bacterial taxa within the community) (FIG. 2a). Sixteen of the peptides significantly increased the Bacteroidetes/Firmicutes ratio in the culture, while three peptides significantly decreased the ratio (FIG. 2b). From a pairwise comparison of the activities of the different cyclic peptides, we found six distinct peptide clusters, each of which affected the microbiota differently from the other clusters. We next carried out in vitro MIC assays against nine representative gut bacterial strains (ranging from high to low abundance in the gut) to determine the effects of the peptides on the growth of bacteria in isolation. The goal of these MIC assays was (i) to validate the effects of the peptides on bacterial growth observed in the en masse assay, (ii) to assess the usefulness of the MIC assays against a panel of representative commensal strains (as compared to our en masse community-based screening) for identifying microbiota remodeling compounds, and (iii) to help inform our selection of lead peptides for in vivo validation. As expected, the peptides showed distinct antibacterial activity profiles against isolated species in the MIC assay, but the growth modulating activities and pairwise clustering of peptide activities did not correlate with those from the en masse screen. This suggests that the tactic of combining data from multiple isolated MIC assays might not suffice for modeling growth-modulating effects in the context of the overall community.

We used a distance scoring algorithm for a more refined compound selection, in which peptides that remodel the microbiota community most completely from the dysfunctional state (WD fed) toward the functional state (CHD fed) are scored higher. Briefly, we first eliminate cyclic D,L-α-peptides that have broad-spectrum antibacterial activity (inhibit >95% of growth of ≥25% of species in the community). The remaining compounds are then ranked based on how well they suppressed the bacterial species that grew in abundance in WD-fed mice. We found that the abundance of 54 bacterial genera significantly changed in LDLr^{-/-} mice fed CHD vs. WD (Table 1). Among these, 40 genera decreased while 14 genera increased in abundance by WD feeding. Within the increased 11 genera, there were 19 bacterial species that were also present in our in vitro culture system. Accordingly, compounds were scored highly when they reduced the levels of 19 bacterial species (lowest summed differences in relative abundance) that were increased by WD feeding to be more similar to their levels in a CHD-fed microbiome (see Table 3 for peptide ranking). Based on these analyses, we chose two peptides for further in vivo validation and efficacy studies: the cationic peptide 1, c[wLwKhShK] (SEQ ID NO:1), and the neutral peptide 11, c[wLwReQeR] (SEQ ID NO:11), which ranked high within their respective peptide clusters.

TABLE 3
Ranking of peptides in remodeling WD gut
microbiota toward CHD microbiota composition
determined by the in vitro en masse screening assay.

| | SEQ | peptide | ranked |
|---|---|---|---|
| | ID | activity | distance |
| peptide | NO | group | to chow |
| WlWlWkSk | 9 | III | 1 |
| wLwKhShK | 1 | I | 2 |
| WwLlHsKk | 4 | II | 3 |
| Chm- 2 μg | N/A | V | 4 |
| LlWhQk | 6 | III | 5 |
| wLfKwKkK | 2 | I | 6 |
| lLwHoK | 24 | V | 7 |
| lFlAlKhK | 10 | III | 8 |
| wLeLwKsK | 16 | III | 9 |
| wLlWkKkS | 5 | III | 10 |
| wLhLwKrK | 21 | IV | 11 |
| wWwKsKsK | 8 | III | 12 |
| YlYlYkSo | 14 | III | 13 |
| wLwReQeR | 11 | III | 14 |
| WwQoHdKt | 27 | VI | 15 |
| wLwSeQsO | 28 | VI | 16 |
| YwElYsKq | 26 | VI | 17 |
| FwHlYoHq | 12 | III | 18 |
| wFkSkSkS | 3 | I | 19 |
| WllLlKkKs | 7 | III | 20 |
| wLlEeKkN | 29 | VI | 21 |
| wLwSeQhK | 25 | VI | 22 |
| YlWyKhAe | 13 | III | 23 |
| Chm- 8 μg | N/A | V | 24 |
| fWwYqHhQ | 15 | III | 25 |

“Peptide activity group” refers to the six groups (clusters) of peptides that were observed in a pairwise comparison of peptide activity in remodeling gut microbiota composition as described in Example 3. Chm, chloramphenicol.

### Example 4 In Vivo Remodeling of the Gut Microbiome

We carried out a 10-week study of cyclic peptides c[wLwKhShK] (SEQ ID NO:1) and c[wLwReQeR] (SEQ ID NO:11) vs. vehicle-treated WD and CHD controls in LDLr^{-/-} mice to determine the safety and effects of the peptides on the gut microbiota and on atherosclerosis. The abiotic structures of cyclic D,L-α-peptides renders them proteolytically stable, allowing oral administration (ad libitum in the drinking water, ˜35 mg/kg/day peptide dose). Importantly, there were no signs of discomfort or toxicity (body weight, liver and spleen weight, AST and ALT enzyme levels), even after a 10-week daily-dosing regimen. As predicted from the in vitro screen, the Bacteroidetes/Firmicutes ratio increased (within 2 weeks by peptide c[wLwKhShK] (SEQ ID NO:1) and within 6 weeks by peptide c[wLwReQeR] (SEQ ID NO:11)) in treated animals relative to the vehicle WD-fed group. The two lead peptides initially targeted different bacterial genera, but over the course of the 10-week study, the remodeled gut microbiome induced by the peptides became more similar. Although the overall microbiota alpha diversity was unchanged by peptide treatment, the microbiota populations in treated animals were distinct from untreated animals. At the species level, the changes in microbiota abundance were also generally similar in vitro and in vivo—9 of the 14 species identified in both the in vitro assay and in vivo study were affected in the same way. Consistent with the in vitro screen, neutral peptide c[wLwReQeR] (SEQ ID NO:11) acted more selectively in vivo against WD-remodeled gut bacteria than the cationic peptide c[wLwKhShK] (SEQ ID NO:1). We conducted a pharmacokinetic study involving i.p. and p.o. administration to support the gut being the site of action. A 16-mg/kg dose of c[wLwReQeR] (SEQ ID NO:11) in mice (BALB/c, n=3) via i.p. administration showed a plasma half-life of ˜6 h and peak plasma concentration of 13 μM, whereas the peptide was not detectable in the plasma of mice (n=3) after oral gavage (limit of detection=3 nM) (Table 4), indicating that the peptides are not absorbed from the digestive tract and act directly within the gut.

TABLE 4
Pharmacokinetic data for c[wLwReQeR]
(SEQ ID NO:11) via different routes of administration.

| | | Route | | | |
|---|---|---|---|---|---|
| | | intraperitoneal | intraperitoneal | subcutaneous | oral gavage |
| Vehicle | | 95% 50 mM | phosphate- | 95% 50 mM | 95% 50 mM |
| | | acetate buffer | buffered | acetate buffer | acetate buffer |
| | | containing | saline | containing | containing |
| | | 10% sucrose, | containing | 10% sucrose, | 10% sucrose, |
| | | 5% DMSO, | 10% | 5% DMSO, | 5% DMSO, |
| | | pH = 24 | sucrose | pH = 4 | pH = 4 |
| Mouse strain | | C57B1/6 | Balb/C | C57B1/6 | C57B1/6 |
| Dose | (mg/kg) | 7.5 | 16 | 7.5 | 15 |
| Cmax | (nM) | 1,133 | 12,800 | 530 | <3 |
| Tmax | (h) | 0.5 | 0.5 | 0.5 | <3 |
| AUC 0-24 h | (nM h) | 3,469 | 51,751 | 1,810 | <3 |

Cmax = maximum observed concentration, Tmax = timepoint at which maximum concentration was observed. n = 3 animals per timepoint. The lower limit of quantitation was 3 nM.

TABLE 5
Observed levels of bile acids in feces and plasma of LDLr^{-/-} mice (n = 7-8 per group)
following 2-wk of treatment, as determined by targeted metabolomics.

| | | | WD + | WD + | |
|---|---|---|---|---|---|
| | bile acid | CHD | vehicle | c[wLwReQeR] | classification |
| Feces | CA | 2300 ± 3000 | 640 ± 570 | 440 ± 490 | primary |
| (pmol/mg | α-MCA | 20 ± 30 | 38 ± 13 | 21 ± 8.9 | primary |
| feces) | β-MCA | 34 ± 22 | 110 ± 30 | 65 ± 12 | primary |
| | ω-MCA | 160 ± 91 | 240 ± 63 | 170 ± 100 | primary |
| | HCA | 0.82 ± 0.39 | 2.9 ± 1.1 | 1.6 ± 1.1 | primary |
| | LCA | 160 ± 65 | 560 ± 280 | 400 ± 170 | secondary |
| | UDCA | 24 ± 14 | 79 ± 29 | 43 ± 16 | secondary |
| | DCA | 4800 ± 1700 | 5000 ± 820 | 4900 ± 2400 | secondary |
| | T-CA | 15 ± 11 | 4.1 ± 1.1 | 8.3 ± 3.3 | conjugated primary |
| | T-α-MCA | 3.8 ± 3.2 | 2.8 ± 0.61 | 17 ± 8.7 | conjugated primary |
| | T-β-MCA | 4.3 ± 3.6 | 3.2 ± 0.71 | 20 ± 11 | conjugated primary |
| | T-ω-MCA | 2.6 ± 2.2 | 0.95 ± 0.21 | 3 ± 1.4 | conjugated primary |
| | T-HCA | 0.080 ± 0.03 | 0.078 ± 0.02 | 0.097 ± 0.03 | conjugated primary |
| | | | | | conjugated |
| | T-DCA | 6.3 ± 7.5 | 0.67 ± 0.18 | 2.6 ± 1.2 | secondary |
| Plasma | CA | 12000 ± 13000 | 2200 ± 1400 | 6300 ± 8300 | primary |
| (pmol/mL | α-MCA | 12 ± 9 | 8.6 ± 3.1 | 10 ± 5.8 | primary |
| plasma) | β-MCA | 74 ± 51 | 42 ± 25 | 76 ± 75 | primary |
| | ω-MCA | 63 ± 27 | 37 ± 12 | 33 ± 11 | primary |
| | HCA | 1.5 ± 1.3 | 2.4 ± 2.2 | 1.0 ± 0.84 | primary |
| | LCA | 81 ± 30 | 96 ± 36 | 100 ± 48 | secondary |
| | UDCA | 160 ± 84 | 100 ± 37 | 120 ± 92 | secondary |
| | DCA | 2600 ± 1600 | 1200 ± 470 | 2100 ± 2100 | secondary |
| | T-CA | 29000 ± 49000 | 1800 ± 780 | 16000 ± 31000 | conjugated primary |
| | T-α-MCA | 1200 ± 2000 | 330 ± 230 | 1900 ± 3900 | conjugated primary |
| | T-β-MCA | 6600 ± 12000 | 900 ± 590 | 9800 ± 22000 | conjugated primary |
| | T-ω-MCA | 2400 ± 3900 | 500 ± 360 | 2400 ± 4900 | conjugated primary |
| | T-HCA | 32 ± 44 | 13 ± 6.2 | 62 ± 110 | conjugated primary |
| | | | | | conjugated |
| | T-DCA | 1200 ± 1700 | 290 ± 110 | 830 ± 1100 | secondary |

### Example 5 Transcriptional Reprogramming of Gut Microbiota and Metabolic Effects

We undertook a comparative analysis in the CHD, WD, and c[wLwReQeR] (SEQ ID NO:11)-treated WD mice of microbiota gene expression level changes to gain insight into the functional consequences of WD-induced and peptide-treated gut microbiome remodeling. Peptide treatment induced widespread reprogramming of the microbiota transcriptome, as determined by RNA-Seq of bacterial RNA from the feces following a 2-week treatment period. The observed gene expression differences (out of ˜53,800 total annotated bacterial genes) could be grouped into three main clusters. Two clusters contained bacterial genes for which expression was increased (Cluster 1) or decreased (Cluster 2) by peptide treatment. Cluster 3 contained genes that were altered by the WD, but not affected by peptide treatment. The majority of bacterial species in the feces sample contained genes present in all three clusters, indicative of pervasive changes not only to the structure, but also the transcriptional activities of the microbiota. Interestingly, overall gene expression from Firmicutes was reduced and overall gene expression from Bacteroidetes was increased by c[wLwReQeR] (SEQ ID NO:11) treatment (Clusters 1 compared to Cluster 2 and 3). These data support the notion that it is not only the relative ratio of bacterial species in the microbiome, but also their overall gene expression levels that govern the function of the gut microbiota.

At the metabolic level, peptide treatment restored important functions of the gut microbiota that were impaired by WD, as determined by functional pathway analysis of metatranscriptomics data and targeted metabolomics. Metabolism of short-chain fatty acids (SCFAs) and amino acids were notable. SCFAs are generated by bacterial fermentation of carbohydrates in the gut and are involved in several microbiota-mediated effects on host metabolism and physiology. Functional metagenomics analyses suggested that peptide treatment induced changes in enzyme expression levels that would lead to increased butyrate biosynthesis. Treatment with c[wLwReQeR] (SEQ ID NO:11) led to higher acetoacetate-CoA transferase levels and lower butyryl-CoA dehydrogenase levels compared to vehicle, both of which would cause a build-up of butyrate. Indeed, targeted metabolomics analysis of fecal samples taken after a two-week treatment period confirmed that animals treated with peptide c[wLwReQeR] (SEQ ID NO:11) had increased levels of fatty acids containing 4-6 carbons, including butyrate. In addition, functional pathway analysis predicted more active catabolism of tryptophan and phenylalanine in WD vehicle animals compared to CHD or WD peptide-treated animals. Indeed, targeted metabolomics revealed higher levels of tryptophan and phenylalanine in the feces of peptide-treated animals after a two-week course. Altogether, our studies highlight the pleiotropic impact of peptide-induced remodeling on transcriptomic and metabolic reprograming of the gut microbiome.

### Example 6 Anti-Atherogenic Effects of Peptide Treatment in WD-Fed LDLr^{-/-} Mice

Plasma cholesterol levels and atherosclerotic lesions were strikingly reduced in the mice on WD following peptide treatment. By the two-week time point, oral administration of peptides c[wLwKhShK] (SEQ ID NO:1) and c[wLwReQeR] (SEQ ID NO:11) had reduced the mouse plasma total cholesterol by 37% and 36%, respectively, compared to the untreated WD group. There was no analogous peptide-mediated reduction in cholesterol in the CHD mice. The reductions in total cholesterol stemmed from marked reduction in the levels of VLDL and LDL, while HDL-cholesterol levels were higher in the peptide-treated animals. We confirmed that peptide treatment did not reduce absorption of dietary cholesterol by carrying out a fecal dual-isotope cholesterol absorption assay. Eight microbiota taxa showed a significant positive correlation with plasma cholesterol levels, while five taxa were negatively correlated. Intriguingly, the peptides generally decreased the abundance of the positively correlated taxa and increased the abundance of negatively correlated taxa. For example, peptide treatment increased the abundance of Bifidobacterium, which was negatively correlated with plasma cholesterol, consistent with previous report of Bordoni et al., Appl. Microbiol. Biotechnol. 97, 8273-8281, 2013. At the completion of the 10-week study, analysis of the whole aorta lesion area indicated striking reductions in atherosclerotic lesions of 37% and 48% by peptides c[wLwKhShK] (SEQ ID NO:1) and c[wLwReQeR] (SEQ ID NO:11), respectively. Similarly, the aortic sinus lesion volume was significantly reduced by 44% and 37%, respectively. Altogether, these studies substantiate a direct link between the state of gut microbiome and development of atherosclerosis and strongly support the viewpoint that directed in vivo remodeling of a dysfunctional gut microbiome can be an effective approach for reducing disease progression.

### Example 7 Gut Microbiota Remodeling Induced Changes in Host Gene Expression

A central question is how remodeling of the gut microbiome induced such significant phenotypic changes in the host. Analysis of mouse liver and ileum (small intestine) samples by RNA-seq revealed that c[wLwReQeR] (SEQ ID NO:11) treatment restored expression of many genes that were downregulated or upregulated in the WD-fed group compared to CHD-fed animals. Most notably, gene ontology analysis indicated that lipid metabolism was upregulated and inflammation- and immune-related responses were downregulated following peptide treatment. Expression of cholesterol 7 alpha-hydroxylase (Cyp7a1), the rate-limiting enzyme in the bile acid (BA) biosynthesis pathway, was significantly increased in peptide-treated WD animals, suggesting increased conversion of dietary cholesterol to bile acids as a possible mechanism for the reduced plasma LDL cholesterol levels. Indeed, we found that the total bile acid in the peptide-treated vs. control WD-fed mice was two-fold higher in the feces and three-fold lower in the plasma. This is consistent with CYP7A1 transgenic mice having a ˜2-3 fold increase in total bile acid pool due to increased hepatic cholesterol catabolism, with associated resistance to diet-induced hypercholesterolemia and atherosclerosis.

Cyp7a1 expression is tightly regulated through a feedback control system, in which high levels of dietary cholesterol induce Cyp7a1 expression, while high plasma levels of bile acids act as signaling molecules to inhibit Cyp7a1 expression and bile acid metabolism by activating the farnesoid X receptor (FXR) and the FGF15/19 endocrine axis. Importantly, the gut microbiota modulates FXR signaling in the gut via bile acid metabolism and processing. FXR-deficient mice on an LDLr^{-/-} background were found to have an improved lipid profile when fed a high-fat diet and were protected against diet-induced obesity and atherosclerosis. Consistent with higher expression of Cyp7a1, we observed that the intestinal expression of FXR and its targets, including fibroblast growth factor 15 (FGF15), were downregulated in peptide-treated animals. Furthermore, not only did peptide-mediated remodeling of the gut microbiota alter the total bile acid pool size it also significantly impacted the relative composition and abundance of certain bile acids in fecal and plasma samples. For example, the levels of taurine-conjugated muricholic acids (T-α,β-MCAs), known FXR antagonists, were increased by peptide treatment, as revealed by targeted metabolomics. Likewise, the expression of intestinal bile acid transporters Asbt, Mrp2, and Mrp3 were significantly altered by the peptide treatment. Together, these data suggest that downregulation of the FGF15 endocrine axis (resulting from the altered processing of bile acids by the remodeled gut microbiota), along with decreased absorption of primary bile acids in the ileum, is likely responsible for maintaining an upregulated bile acid metabolism in the liver, resulting in increased conversion of cholesterol to bile acids that contributes to lower plasma cholesterol levels.

### Example 8 Anti-Inflammatory and Immunomodulatory Effects

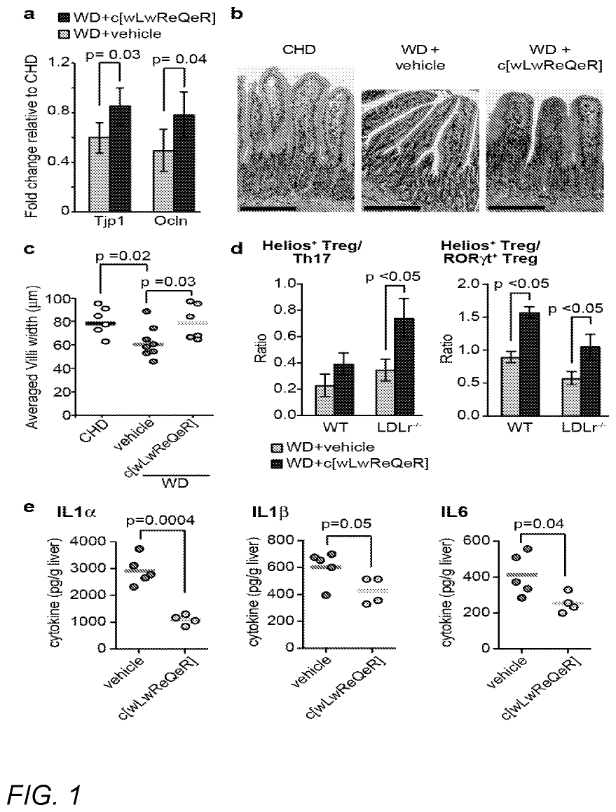

Inflammation plays a critical role in the development of atherosclerosis. We found that peptide-induced remodeling of the gut microbiome reduced the inflammation caused by WD via several gut microbiota-dependent mechanisms. Some commensal gut bacteria play key roles in the maintenance of gut integrity. Impaired gut integrity can lead to increased leakage of microbiota-derived lipopolysaccharide (LPS) from the gut, causing systemic inflammation that intensifies atherosclerosis pathogenesis. We found that several genes related to tight junctions in the ileum were upregulated by peptide treatment (FIG. 1a), and LPS-lipopolysaccharide response pathways were downregulated, suggesting that the defects in gut integrity caused by a WD regimen had been largely mitigated. Indeed, the villi width in the ileum (although not the length) was significantly increased in peptide-treated animals compared to vehicle controls (FIG. 1b,c).

We found that peptide-induced gut microbiome remodeling also impacted the immune cell composition in the gut. Helios⁺ thymic derived regulatory T cells (tTregs) regulate key pathways for preventing uncontrolled inflammatory responses and play important role in inhibiting the development of atherosclerosis in multiple mouse models. Furthermore, the differentiation and maintenance of different subsets of Tregs in the intestine are driven by signals from the gut microbiota. We found that the composition of Foxp3⁺ regulatory T cell (Treg) population was significantly altered by c[wLwReQeR] (SEQ ID NO:11) treatment (FIG. 1d). We isolated cells from the lamina propria tissue and measured the relative populations of Helios⁺ Foxp3+ Tregs to two other subsets of T cells: RAR-related orphan receptor γt (RORγt⁺ Foxp3⁺) Tregs and to proinflammatory RORγt⁺ T helper 17 (Th17) cells. Peptide treatment increased the population of anti-inflammatory Helios⁺ Tregs in both wild-type and LDLr^{-/-} mice fed a WD, as indicated by increased Helios⁺/Th17 ratio and increased Helios⁺/RORγt⁺ Treg ratio (FIG. 1d). It is interesting to speculate that since a number of oxysterols serve as natural endogenous RORγt agonists, the observed reduction in proinflammatory RORγt⁺ Th17 cells could be related to the altered profile of oxysterols and bile acids resulting from the peptide-induced remodeling of the gut microbiota.

Interleukin-1β (IL-1β) is a proinflammatory cytokine central to the inflammatory response driven by the interlukin-6 (IL-6) signaling pathway. Inhibition of IL-1β has been shown to reduce the development of atherosclerotic plaques in mice and significantly lower the incidence of recurrent cardiovascular events in humans. In addition to IL-1β, a pathogenic role of IL-la in vascular inflammation and atherosclerosis has been described. Analysis of liver tissue from WD animals (peptide c[wLwReQeR] (SEQ ID NO:11) treated vs vehicle) using a Luminex assay showed significant reductions in several cytokine and chemokine levels, including IL-1β, IL-1α, IL-6, and TNF-α (FIG. 1e, Table 6), providing further support for the systemic anti-inflammatory effects of peptide-induced remodeling of a dysfunctional gut microbiome.

TABLE 6
Observed levels of cytokines/chemokines in liver tissue of WD-fed
LDLr^{-/-} mice following 4 weeks of treatment with c[wLwReQeR]
(SEQ ID NO:11) or vehicle, as determined by Luminex assay.

| | | WD + | |
|---|---|---|---|
| Cytokine/ | WD + vehicle | c[wLwReQeR] | |
| chemokine | (pg/g tissue) | (pg/g tissue) | p value |
| CCL2 (MCP-1) | 1100 ± 350 | 470 ± 150 | 0.02 |
| CCL2 (MIP-2) | 1000 ± 290 | 460 ± 110 | 0.01 |
| CCL3 (MIP-1a) | 2200 ± 1900 | 200 ± 130 | NS |
| CCL4 (MIP-1b) | 17000 ± 9400 | 3000 ± 1800 | 0.02 |
| CXCL10 (IP-10) | 2100 ± 590 | 1200 ± 380 | 0.04 |
| Eotaxin | 1700 ± 1100 | 1200 ± 390 | NS |
| G-CSF | 380 ± 55 | 380 ± 150 | NS |
| GM-CSF | 140 ± 88 | 86 ± 37 | NS |
| IFNg | 56 ± 25 | 37 ± 16 | NS |
| IL-12p40 | 270 ± 100 | 200 ± 78 | NS |
| IL-12p70 | 270 ± 42 | 270 ± 140 | NS |
| IL-15 | 840 ± 140 | 960 ± 370 | NS |
| IL-17 | 67 ± 12 | 55 ± 16 | NS |
| IL10 | 170 ± 44 | 160 ± 58 | NS |
| IL13 | 5.9 ± 4.2 | 7.3 ± 5.9 | NS |
| IL1a | 2900 ± 540 | 1100 ± 190 | 0.0004 |
| IL1b | 610 ± 130 | 430 ± 100 | 0.05 |
| IL2 | 120 ± 32 | 80 ± 24 | NS |
| IL3 | 34 ± 8 | 31 ± 15 | NS |
| IL4 | 13 ± 2.3 | 13 ± 8.4 | NS |
| ILS | 40 ± 9.1 | 39 ± 25 | NS |
| IL6 | 410 ± 120 | 260 ± 55 | 0.04 |
| IL7 | 220 ± 52 | 230 ± 110 | NS |
| IL9 | 1100 ± 240 | 670 ± 160 | 0.01 |
| KC | 450 ± 200 | 190 ± 20 | 0.04 |
| LIF | 8.6 ± 2.8 | 7.1 ± 4 | NS |
| LIX | 320 ± 370 | 290 ± 240 | NS |
| M-CSF | 260 ± 50 | 200 ± 70 | NS |
| MIG | 1800 ± 660 | 1200 ± 650 | NS |
| RANTES | 170 ± 110 | 71 ± 61 | NS |
| TNFa | 110 ± 20 | 77 ± 21 | 0.03 |
| VEGF | 13 ± 4.2 | 8.2 ± 3.4 | NS |

n = 4-5/group

### Example 9 Materials and Methods

Animals and diets. LDL receptor-null (LDLr^{-/-}) mice on a C57BL/6J background were initially purchased from Jackson Laboratories (Bar Harbor, Me.) and were bred in house. Unless otherwise noted, all experiments were performed with 8-12 weeks old LDLr^{-/-} female mice. For studies involving analysis of the gut microbiome, the mice were rotated between cages and co-housed with the mice from different groups for at least one week before the beginning of each study to reduce the variation in the composition of gut microbiome caused by the housing environment. The mice were weaned at 4 weeks of age and were fed ad libitum a standard chow diet (Harlan Teklad 7019) until they were 10-weeks old, when they were switched to a high-fat diet (WD) containing 15.8% (wt/wt) fat, 1.25% (wt/wt) cholesterol, and no cholate (Harlan Teklad 94059). At the time that the WD was started, the cyclic peptide was added to the drinking water at a dose of 180 μM and the mice (female, n=8 per dosing group) were continued on WD for 10 weeks. The lyophilized peptide was dissolved in PBS (pH 7.4) containing 1% sucrose, resulting in a clear solution. The drinking water solution was replaced with fresh solution daily for c[wLwReQeR] (SEQ ID NO:11) or every other day for c[wLwKhShK] (SEQ ID NO:1). A control group of eight mice (female, n=8) was fed WD and provided vehicle (PBS with 1% sucrose) drinking water. The mice consumed approximately 4.0 mL of water per day per mouse, and there was no significant difference in water or food consumption between groups. All procedures involving live animals were approved by the Scripps Research Institute Institutional Animal Care and Use Committee.

Cyclic peptide synthesis and preparation. Peptides were synthesized by using standard Fmoc chemistry with an Advanced Chemtech Apex 396 peptide synthesizer. A typical synthesis was performed on 0.09-mmol scale using 0.6 mmol/g Rink amide MBHA resin loaded with Fmoc-Glu-OAll via the side chain to yield a Gln residue after cleavage from the resin, or loaded with Fmoc-Lys-OAll via the side chain. Standard side chain protecting groups included Gln(Trt), Asn(Trt), Lys(Boc), Orn(Boc), diaminopropionic acid(Boc), diaminobutyric acid(Boc), His(Trt), Ser(tBu), Trp(Boc), Glu(OtBu). Chain elongations were achieved using 1,3-diisopropylcarbodiimide (DIC) and HOBt in N-methylpyrrolidin-2-one (NMP) with 90-min couplings. Fmoc deprotection was achieved using 25% piperidine in NMP. After full elongation of the peptide, the terminal Fmoc was removed, and then the allyl group was cleaved using 0.5 mol-equiv of Pd(PPh₃)₄ with 10 mol-equiv of PhSiH₃ in CH₂Cl₂ (2×1.5 h). The peptides were cyclized on resin using bromotripyrrolidinophosphonium hexafluorophosphate (PyBroP) (5 mol-equiv) and i-Pr₂NEt (12 mol-equiv) in DMF for 2 h. Peptides were cleaved from the resin with concomitant side chain deprotection by agitation in a solution of 95:2.5:2.5 TFA:triisopropylsilane (TIS):water for 3 h. The peptide was precipitated with ether, centrifuged, and washed three additional times with ether. The crude peptides were purified by preparative reverse-phase (RP)-HPLC on a Vydac 218TP C18 or Thermo BioBasic C18 column. Purity was confirmed by analytical RP-HPLC. Purified peptides were characterized by analytical HPLC and LC-mass spectrometry. Analytical RP-HPLC was performed using a Zorbax 300-SB C-18 column connected to a Hitachi D-7000 HPLC system. Binary gradients of solvent A (99% H₂O, 0.9% acetonitrile, 0.1% TFA) and solvent B (90% acetonitrile, 9.9% H₂O, 0.07% TFA) were employed for HPLC.

In vitro cecum culture and peptide screening. We screened a number of parameters to optimize the in vitro culture condition for maximal bacterial diversity in liquid culture, including a survey of several different growth media, dilution factors for plate inoculation, and incubation times; further details of the development of this in vitro screen will be reported elsewhere. Female LDLr^{-/-} mice (8-10 weeks of age) were fed a WD for two weeks. Cecum harvest and all assay steps were performed under anaerobic conditions (Coy Laboratory anaerobic chamber). Cecum contents from three mice were pooled for the assay to provide enough material and to minimize the individual variation in gut microbiome. Cecum contents were suspended in PBS (1.5 ml PBS per 100 mg of cecum content). The mixture was vortexed for 5 min and the supernatant was transferred to a new tube. The cecum content mixture was further diluted in Chopped Meat Carbohydrate Broth (BD 297307) for in vitro peptide screening. We found that Chopped Meat Carbohydrate broth maintained higher bacterial diversity during a 48-hour in vitro culture compared to other media that were examined. The concentration of peptide stock in DMSO was calculated based on A280 measurement. For each peptide, an initial 20 mM stock was made in DMSO and diluted to 1 mM in 10% sucrose. From that, a 0.64 mM peptide dilution in 10% sucrose was prepared, and each peptide was further diluted into the assay for peptide screening at two concentrations (64 μM and 16 μM). Bacterial DNA was harvested after 24-hour peptide treatment and isolated with Microbial DNA isolation kit (Mo Bio) following the manufacturer's instructions.

Feces sample collection and DNA extraction. The feces samples were freshly collected after 2, 6, and 10 weeks of treatment, snap-frozen, and stored at −80° C. Microbial genomic DNA was isolated with PowerSoil DNA isolation kit by following manufacturer's instructions (Mo Bio).

16S rRNA gene sequencing and processing. PCR amplicons of the V3-V4 16S rRNA region were amplified and sequenced using an Illumina MiSeq platform. For each sample, duplicate 25 μl PCR reactions were performed, each containing 50 ng of purified DNA, 12.5 μl 2×KAPA HiFi HotStart ReadyMix (Kapa biosystem), and 0.15 μM of each primer designed to amplify the V3-V4 region:

forward primer
(SEQ ID NO: 30)
(5′-AATGATACGGCGACCACCGAGATCTACACTCTTTCCCTACACGACG
CTCTTCCGATCTNNNNNNNNNNNNACTCCTACGGGAGGCAGCAG-3′)
and
reverse primer
(SEQ ID NO: 31)
(5′-CAAGCAGAAGACGGCATACGAGATNNNNNNNNNNNNGTGACTGGAG
TTCAGACGTGTGCTCTTCCGATCTnnnnnnnnnnGGACTACHVGGGTWTC
TAAT-3′).

A unique 12-base sequence (N's) was included for sample identification. We also included unique molecular identifiers (n's) to correct PCR duplication artifacts during the library preparation. Cycling conditions were 98° C. for 2 min, followed by 20 cycles of 98° C. for 10 s, 60° C. for 30 s, and 72° C. for 45 s. A condition of 72° C. for 5 min was used for the final elongation step. Replicate PCR samples were pooled, and amplicons were qualified with Quant-iT dsDNA Assay kit (ThermoFisher). Each sample was combined at an equimolar amount before DNA purification with Agencourt AMPure XP (Beckman Coulter). The completed library was sequenced on an Illumina MiSeq platform following the procedures recommended by Illumina.

16S rRNA gene sequencing data analysis. We used custom python scripts to demultiplex sequencing reads based on the second barcode index and remove PCR duplication artifacts found using unique molecular identifiers prior to analyzing sequences using MOTHUR (version 1.36.1). We developed a custom analysis pipeline based on the MiSeq SOP published by the curators of MOTHUR. Briefly, contigs were generated by combining forward and reverse paired-end reads. The contigs were filtered based on their length (440-480 bp for V3-V4 region), and any contigs with ambiguous bases were removed. The unique contigs were then aligned to a reference database (SILVA non-redundant dataset v119). The contigs that didn't align properly based on position of alignment and those with homopolymers of length >8 were removed before the downstream analysis. We performed a pre-clustering step to cluster reads up to 2 bp apart and removed all chimeric sequences. Finally, we used a k-mer based method to classify the sequences to individual taxonomic groups to get counts per group.

Measurement of plasma cholesterol and liver enzyme activities. The mice were bled after an overnight fast after 2 and 10 weeks of treatment. The plasma samples were used to determine lipoprotein profiles and the levels of cholesterol, alanine aminotransferase (ALT), and aspartate aminotransferase (AST). Measurements of plasma levels of cholesterol, ALT, and AST were performed as described in Zhao et al., ACS Cent. Sci. 3, 639-646, 2017. Briefly, plasma was separated from whole blood immediately by centrifugation of the blood samples at 5,000 rpm for 10 min at 4° C., and was then stored at −80° C. Plasma total cholesterol was measured using an enzymatic fluorometric method kit (Amplex® red cholesterol assay kit, No. A12216, Life Technologies) according to the manufacturer's instructions. Plasma ALT and AST concentrations were measured using Infinity ALT (GPT) and AST (GOT) liquid stable assays, respectively, which are colorimetric kinetic assays (Thermo Scientific). Assays were performed in accordance with the manufacturer's recommendations, adjusting the reagent volumes (20 μL plasma+200 μL reagent, 0.69 cm light pathlength of the solution in the well) for analysis of samples in 96-well flat-bottom microplate format.

Pharmacokinetics. These experiments were carried out in part courtesy of Carol S. Ryan, Debra J. Search, Ricardo A. Garcia, and David A. Gordon at Bristol Myers Squibb (Hopewell, N.J., USA). Male Balb/C or C57BL/6 mice (20 g) were maintained on a chow diet. The cyclic peptide c[wLwReQeR] (SEQ ID NO:11) was dissolved in PBS containing 1% sucrose or 95% 50 mM acetate buffer containing 10% sucrose, 5% DMSO (pH=4), and sterile filtered through a 0.22-μm syringe filter before injection. Mice were fasted beginning 12 h before dosing, and continued the fast until after the 8-h time point blood draw was completed. Groups of three mice received a dose of the cyclic peptide via intraperitoneal injection (5 mL/kg for subcutaneous and intraperitoneal, 10 mL/kg for oral gavage). Blood was drawn (30-60 μL) from the retro-orbital sinus into a heparinized capillary tube before dosing (0 min) and at different intervals from 30 min to 8 h after dosing. Plasma was isolated immediately from the whole blood by centrifugation at 5,000 rpm for 10 min at 4° C. Immediately after the plasma was isolated, 20 μL of plasma was acidified with 20 μL of 5% TFA to break peptide-protein interactions, and 40 μL of acetonitrile was then added. After vortexing for 30 s, the mixture was centrifuged at 13,000 rpm for 10 min at 4° C. The resultant supernatant was analyzed by using LC-MS SIM as described below.

LC-MS SIM quantitation of peptide concentration. Cyclic peptide concentrations were quantified by using reverse-phase HPLC coupled with mass spectrometry. The electrospray ionization mass spectrometry measurements were carried out in the positive ionization mode using a single quadrupole mass spectrometer (Hewlett Packard HP 1100 MSD series). 10 or 20 μL of sample were injected through a C8 reverse-phase column (Zorbax 300-SB, 4.6 mm×150 mm, 5 μm) using a flow rate of 1.5 mL/min and binary gradients of solvent A (99% H₂O, 0.1% formic acid, 0.01% TFA) and solvent B (99% acetonitrile, 0.1% formic acid, 0.01% TFA). Mass detection was carried out in the selected ion monitoring (SIM) mode for the positive molecular ion, with the optimized fragmentor and capillary voltages of 180 V and 5 kV, respectively. The selected monitoring mass for cyclic peptide c[wLwReQeR] (SEQ ID NO:11) was 592.8 ([M+2H]²⁺). For quantitative calibration, standard curves were established using mouse EDTA-anticoagulated plasma spiked with various concentrations of the peptide. The calibration curve was established by using linear fitting of the data, with correlation coefficient ≥0.98.

In vivo cholesterol absorption assay. This experiment was carried out courtesy of Carol S. Ryan, Debra J. Search, Ricardo A. Garcia, and David A. Gordon at Bristol Myers Squibb (Hopewell, N.J., USA). To assess the effects of c[wLwReQeR] (SEQ ID NO:11) on intestinal absorption of cholesterol, male LDLr^{-/-} mice (Jackson Laboratories) ranging from 8-10 weeks old were acclimated to single housing in standard solid bottom bedded cages for at least one week at BMS facilities. During the acclimation period, the mice were fed a chow diet ad libitum. At the start of the treatment period and for the duration of the study, mice were switched to high-fat, high-cholesterol Western diet (Research Diets D12108C) containing 1.25% cholesterol and 20% fat ad libitum. The peptide was administered in the drinking water ad libitum as a solution with 1% sucrose at 180 μM (˜35 mg/kg/day). As a positive control, ezetimibe (3 mg/kg) was administered by once daily oral gavage at 5 mL/kg in a formulation containing 0.1% Tween 80 and 0.75% carboxymethyl cellulose.

Following a 2-wk treatment period, cholesterol absorption was measured using a dual fecal isotope ratio method. Mice were fasted for 4 hours and each animal was administrated 50 μL of radioisotope cocktail composed of the following: 1.0 μCi of [¹⁴C]-cholesterol and 2.0 μCi of μ-[³H]-sitosterol mixed in corn oil as a carrier. Feces were collected for a period of 48 hours following administration of radioisotope cocktail. At least 50 μl of blood was collected at 24 h and 48 h following administration of radioisotope cocktail via retro-orbital bleeding and was centrifuged to allow isolation of plasma. Plasma (˜20 μl) was added to 5 ml of Opti-fluor Scintillation cocktail (Packard Bioscience #6013199) and counted on ³H and ¹⁴C channels to evaluate cholesterol efflux. Radioactivity in feces was analyzed as follows: for each animal, feces recovered over the 48 hours was dried overnight at 50° C. and 0.1 gram of dried feces was transferred to a glass tube. The feces were dissolved in 0.7 ml water and vortexed to a paste. The sample was then treated with 1.4 ml of 1 N NaOH in EtOH at 85° C. for 2 hours. Lipids were extracted with 3 ml of petroleum ether, and samples were centrifuged at 600 rpm for 5 minutes. A volume of 2.0 ml of the top layer was transferred to a scintillation vial. The samples were dried under a stream of nitrogen gas, and then reconstituted with 100 μl chloroform/methanol (1:2). Finally, 5 ml of scintillation fluid was added to the vials for scintillation counting.

Plasma lipoprotein profile. Pooled plasma (240 μL total, 30 μL from each, n=8 fasted mice per group) from the two-week blood draw was used for fast protein liquid chromatography (FPLC) analysis. Lipoproteins were separated by using 3 Superdex 200 10/30 columns connected in series (GE Healthcare). The plasma was centrifuged at 11,000 rpm for 10 min at room temperature to remove particulates (floating material was gently mixed into liquid before removing the supernatant for FPLC injection). 200 μL of the pooled plasma was injected on the system eluted with 10 mM tris-HCl buffer, pH 7.4, containing 1 mM EDTA and 150 mM NaCl; a flow rate of 0.5 ml/min and fraction size of 0.5 mL were used.

Analysis of atherosclerosis. Atherosclerotic lesion severity was assessed in the aortae as previously described. Briefly, at euthanasia, animals were perfused with PBS, followed by 4% formaldehyde (10% UltraPure EM Grade from Polysciences diluted in PBS, pH 7.2). For en face analysis, the entire mouse aorta was dissected from the proximal ascending aorta to the bifurcation of the iliac artery by using a dissecting microscope. Adventitial fat was removed, and the aorta was opened longitudinally, pinned flat onto black dissecting wax, stained with Sudan IV, and photographed at a fixed magnification. The photographs were digitized, and total aortic areas and lesion areas were calculated by using Adobe Photoshop and NIH Image J software. The results were reported as a percentage of the total aortic area that contained lesions.

As a second assessment of atherosclerosis, lesions of the aortic root (heart sinus) were analyzed⁵⁵. Utilizing stereological principles, lesion volume was estimated across a fixed distance of the aortic sinus. After 10 min fixation in 4% paraformaldehyde, hearts were cut at an angle perpendicular to the atria of the heart and embedded in OCT (Tissue-Tek). Frozen hearts were sectioned on a Leica cryostat, with 10-μm sections collected from the beginning of the aortic sinus (defined as when a valve leaflet became visible) to 500 μm below the beginning of the sinus. For hearts cut at an angle that resulted in valve leaflets not appearing in the same section (due to poor section angle), the lagging leaflet was used to determine the 500-μm distance. Sections were collected in duplicate at 50-μm intervals. Sections were stained with oil red O, counterstained with Gill hematoxylin 1 (Fischer Scientific International), photographed, and digitized for lesion analysis. Scoring of valve lesion areas was done for each of the 3 valve cusps individually. Lesion areas found only within the valve cusp were measured. Lesion volume estimation was determined from a 1-in-10 sampling rate; hence, valve cusps spaced at 140 μm were used to determine the lesion volume for a total of four sections analyzed per valve cusp. Lesion volume was calculated from an integration of the measured cross-sectional areas. Prediction of the coefficient of error (CE) in approximating lesion volume was computed using the Cavalieri estimator derived from a covariogram analysis of an ordered set of estimates of cross-sectional areas. This yielded CE values of less than 10% that were acceptable for a stereological computation of lesion volume.

Bacterial RNA-seq sample preparation and sequencing. Total bacterial RNA from feces samples was isolated with ZR Soil/Fecal RNA MicroPrep (Zymo research) following the manufacturer's instructions. Briefly, the sample was suspended in RNA lysis buffer and lysed by the mixer (Retsch). The supernatant was transferred to a RNA-binding column and washed several times with RNA wash buffer. In-column DNaseI digestion at 25° C. for 15 min was performed to eliminate DNA contamination in the sample. The presence of genomic DNA contamination was assessed by PCR with universal 16S rRNA gene primers. Before RNA-seq library preparation, rRNA was removed from 2 μg of total bacterial RNA with Ribo-Zero Bacteria kit (Illumina). 100 ng of purified RNA was used for RNA-seq library preparation as described previously⁵⁶. The cDNA was synthesized by reverse transcription with SuperScript III (Life technologies) and second-strand synthesis (New England Biolabs). The sequencing library was generated from purified cDNA with Nextera XT DNA library preparation kit (Illumina) and amplified by PCR. Cycling conditions were 72° C. for 3 min, 95° C. for 30 s, followed by 16 cycles of 95° C. for 10 s, 55° C. for 30 s, and 72° C. for 30 s. A condition of 72° C. for 5 min was used for the final elongation step. Libraries with different indexes were pooled and single-end sequencing (75 bp) was performed on an Illumina NextSeq at the Scripps Research Institute next generation sequencing core.

Functional analysis of bacterial RNA-seq data. We used BWA (version 0.7.12) read alignment program and a custom-built bacterial reference database, which contains 4,700 bacterial genomes from NCBI NR sequence database, to map RNA-seq reads. The reads aligned to individual bacterial genes were counted using HTSeq count (version 0.6.0) and the counts were further normalized based on size factors estimated using the median of the ratios of observed counts method as described previously (Anders et al., Genome Biol. 11, R106, 2010). The proteins predicted by RNA-seq read alignment were annotated with DIAMOND (version 0.8.26) using the NCBI nonredundant (NCBI-nr) database and the default setting (BLASTX e value <10⁻³, bit score >50). For query genes with multiple matches, the annotated reference gene with the lowest e value was chosen. The functional analysis was performed by MEGAN (version 5.11.3) and KEGG analyzer was used to determine read counts in different metabolic pathways from each group. The aggregate expression level changes for sequences within a given function were normalized before the comparison between each group.

SCFA and amino acid measurements. Fecal SCFA and amino acid content was measured by liquid chromatography-mass spectrometry (LC-MS). The fecal samples were weighed and placed into 2 ml Omni tubes. Ethanol was added to bring the volume to 350 μl. Next, 4 μl of 500 μM d-FA-mix and 20 μl of 1 mM 3CHPA were added to each sample as internal standard. The samples were homogenized using an Omni bead beater at the speed of 4.5 m/s for 30 sec, and then centrifuged at 13,000 rpm for 15 min. 90 μl of the supernatants was transferred into glass HPLC vials. 30 μl of freshly prepared 4×3NPH solution (160 mM 3-nitrophenylhydrazine, 150 mM EDC, pyridine 12% vol, 50% acetonitrile) was added to each sample and the reactions were held at room temperature for 30 min. 2 μl of the sample was injected into the LC-MS (Agilent 1290 HPLC, 6550 qTOF). The concentration of each SCFA and amino acid was calculated based on a standard curve and normalized by the internal control and the feces weight.

Mouse RNA-seq sample preparation and sequencing. Mouse tissues were harvested after 2 weeks of treatment, stored in RNAlater, and snap-frozen. 20-30 mg of mouse tissue was used for RNA extraction with RNeasy Mini Kit (Qiagen) following the manufacturer's instructions. Ribosomal RNA was removed from total RNA with Ribo-Zero Gold rRNA removal kit (Illumina). Strand-specific RNA-seq libraries were constructed as described previously (Hainer et al., Genes Dev. 29, 362-378, 2015). Libraries with different barcodes were pooled and single-end sequencing (75 bp) was performed on an Illumina NextSeq at the Scripps Research Institute next generation sequencing core.

Mouse RNA sequencing data analysis. After ribosomal RNA sequences were removed, the RNA transcripts were quantified using RSEM. Differentially expressed genes were identified by DESeq2 and significantly changed genes were selected using a cutoff of adjusted p-value <0.1, comparing peptide-treated animals (n=3) to vehicle-control animals (n=3) for each group (CHD or WD-fed animals). Enrichment of Gene Ontology terms and categories was performed with DAVID 6.8.

Quantitative real-time PCR. To validate selected gene expression results, qPCR reactions were carried out. Mouse livers were harvested two weeks after peptide treatment and total RNA was extracted using RNeasy Mini (Qiagen) following manufacturer's instructions. Reverse transcription was performed for 1 h using random priming (Promega). qPCR reactions (0.5 μl cDNA, 0.2 μM each primer, SYBR green Master Mix (Kapa biosystems)) were performed on a Bio-Rad CFX384 Touch Real-Time PCR detection system, using primers specific for each gene. Data were normalized to loading controls (16S and β-actin).

Primers for qPCR experiment:

| Gene | Forward (5′-3′) | Reverse (5′-3′) |
|---|---|---|
| name | (SEQ ID NOs: 32-43) | (SEQ ID NOs: 44-55) |
| 16S | CCGCAAGGGAAAGATGAAA | TCGTTTGGTTTCGGGGTTTC |
| | GAC | |
| β-actin | ATGGAGGGGAATACAGCCC | TTCTTTGCAGCTCCTTCGTT |
| Cyp7a1 | AGCAACTAAACAACCTGCCAG | GTCCGGATATTCAAGGATGCA |
| | TACTA | |
| Cyp8b1 | GGCTGGCTTCCTGAGCTTATT | ACTTCCTGAACAGCTCATCGG |
| Cyp27a1 | GCCTCACCTATGGGATCTTCA | TCAAAGCCTGACGCAGATG |
| Aka1d1 | TGCACACCACCAAATATCCCT | CTTCACTGCCACATAGGTC |
| | | TTC |
| Mrp2 | TCTGTGAGTGCAAGAGACA | TCCAGGACCAAGAGATTTGC |
| | GGT | |
| Mrp3 | CCGAAACTACGCACCAGATG | GATGGCTGGCTCATTGTCTG |
| Asbt | TGGTGTAGACGAAGAGGCAA | GCCTATTGGATAGATGGCGA |
| Fgf15 | CAGTCTTCCTCCGAGTAGCG | TGAAGACGATTGCCATCAAG |
| Tjp1 | CCTGTGAAGCGTCACTGTGT | CGCGGAGAGAGACAAGATGT |
| Ocln | CATAGTCAGATGGGGGTGGA | ATTTATGATGAACAGCCCCC |

Bulk bile acid quantification. Total bile acids were measured enzymatically using a total bile acid assay kit (Cell Biolabs) following manufacturer's instruction. Feces were obtained between 0900-1100 am from non-fasted animals after a 2-wk treatment period. The extraction of bile acid from feces was performed as described in Yu et al., J. Biol. Chem. 275, 15482-15489, 2000. In brief, the feces from individual mice were collected, weighed, and agitated in 75% ethanol in an ultrasonic bath at 50° C. for 2 h. After centrifugation at 3,500 g for 10 min, the supernatant was transferred to a new tube and diluted with 25% PBS. The total bile acid level was then determined enzymatically using the kit. For plasma samples, the plasma was diluted 1:10 with 25% PBS prior to enzymatic bile acid measurement using the kit.

Measurement of secondary bile acids. Feces were obtained between 0900-1100 am from non-fasted animals after a 2-wk treatment period. Bile acids were extracted from samples according to published procedures (Wegner et al., Anal. Bioanal. Chem. 409, 1231-1245, 2017). Briefly, fecal samples were lyophilized and homogenized. Five milligrams of fecal powder were extracted with 250 μl of methanol containing heavy internal standards. Fifty microliters of plasma were extracted with 150 μL of methanol containing heavy internal standards. After vortexing for 10 minutes and centrifuging (16,000×g, 4 C, 10 min), supernatants were transferred to glass vials for injection and analysis by LCMS. Bile acids were analyzed on a Dionex Ultimate 3000 LC system (Thermo) coupled to a TSQ Quantiva mass spectrometer (Thermo) fitted with a Kinetex C18 reversed phase column (2.6 μm, 150×2.1 mm i.d., Phenomenex). The following LC solvents were used: solution A, 0.1% formic acid and 20 mM ammonium acetate in water, solution B, acetonitrile/methanol (3/1, v/v) containing 0.1% formic acid and 20 mM ammonium acetate. The following reversed phase gradient was utilized: at a flow rate of 0.2 mL/min with a gradient consisting of 25-29% B in 1 min, 29-33% B in 14 min, 33-70% B in 15 min, up to 100% B in 1 min, 100% B for 9 min and re-equilibrated to 25% B for 10 min, for a total run time of 50 min. The injection volume for all samples was 10 μL, the column oven temperature was set to 50° C. and the autosampler kept at 4° C. MS analyses were performed using electrospray ionization in positive and negative ion modes, with spray voltages of 3.5 and −3 kV, respectively, ion transfer tube temperature of 325° C., and vaporizer temperature of 275° C. Multiple reaction monitoring (MRM) was performed by using mass transitions between specific parent ions into corresponding fragment ions for each analyte. The transitions and retention time for each analyte and internal standards are shown in Table 7.

TABLE 7
MRM transitions in positive and negative modes for all bile acids.

| | Retention | MW | Ionization | MRM |
|---|---|---|---|---|
| Analyte | time (min) | (g/mol) | Mode | transition (m/z) |
| d5-CA | 25.8 | 413.6 | Positive | 431.4 → 378.3 |
| (d5-Cholic Acid) | | | | 431.4 → 360.3 |
| | | | | 431.4 → 396.3 |
| | | | | 431.4 → 342.2 |
| CA | 25.8 | 408.57 | Positive | 426.3 → 355.2 |
| (Cholic Acid) | | | | 426.3 → 373.2 |
| | | | | 426.3 → 391.2 |
| | | | | 426.3 → 337.2 |
| α-MCA | 22.2 | 408.57 | Positive | 426.3 → 355.2 |
| (α-Muricholic Acid) | | | | 426.3 → 373.2 |
| | | | | 426.3 → 391.2 |
| | | | | 426.3 → 337.2 |
| β-MCA | 23 | 408.57 | Positive | 426.3 → 355.2 |
| (β-Muricholic Acid) | | | | 426.3 → 373.2 |
| | | | | 426.3 → 391.2 |
| | | | | 426.3 → 337.2 |
| ω-MCA | 21.8 | 408.57 | Positive | 426.3 → 355.2 |
| (ω-Muricholic Acid) | | | | 426.3 → 373.2 |
| | | | | 426.3 → 391.2 |
| | | | | 426.3 → 337.2 |
| HCA | 24.6 | 408.57 | Positive | 409.4 → 355.2 |
| (Hyocholic Acid) | | | | 409.4 → 373.2 |
| | | | | 409.4 → 337.1 |
| d4-LCA | 33.2 | 380.6 | Negative | 379.3 → 379.3 |
| (d4-Lithocholic Acid) | | | | |
| LCA | 33.2 | 376.57 | Negative | 375.3 → 375.3 |
| (Lithocholic Acid) | | | | |
| d4-UDCA | 25.7 | 396.6 | Negative | 395.3 → 394.8 |
| (d4-Ursodeoxycholic Acid) | | | | 395.3 → 377.3 |
| UDCA | 25.7 | 392.57 | Negative | 391.3 → 390.7 |
| (Ursodeoxycholic Acid) | | | | 391.3 → 377.3 |
| d4-DCA | 30.2 | 396.6 | Positive | 414.3 → 361.2 |
| (d4-Deoxycholic Acid) | | | | 414.3 → 379.2 |
| | | | | 414.3 → 343.2 |
| DCA | 30.2 | 392.57 | Positive | 410.4 → 357.2 |
| (Deoxycholic Acid | | | | 410.4 → 375.3 |
| | | | | 410.4 → 339.2 |
| d4-TCA | 16.7 | 519.7 | Positive | 537.3 → 520.3 |
| (d4-Taurocholic Acid) | | | | 537.3 → 466.3 |
| | | | | 537.3 → 484.2 |
| | | | | 537.3 → 502.2 |
| TCA | 16.7 | 515.70 | Positive | 533.3 → 462.2 |
| (Taurocholic Acid) | | | | 533.3 → 516.3 |
| | | | | 533.3 → 498.2 |
| | | | | 533.3 → 480.2 |
| T-α-MCA | 7.8 | 515.70 | Positive | 533.3 → 462.2 |
| (Tauro-α-Muricholic Acid) | | | | 533.3 → 516.3 |
| | | | | 533.3 → 498.2 |
| | | | | 533.3 → 480.2 |
| T-β-MCA | 8.2 | 515.70 | Positive | 533.3 → 462.2 |
| (Tauro-β-Muricholic Acid) | | | | 533.3 → 516.3 |
| | | | | 533.3 → 498.2 |
| | | | | 533.3 → 480.2 |
| T-ω-MCA | 7.4 | 515.70 | Positive | 533.3 → 462.2 |
| (Tauro-ω-Muricholic Acid) | | | | 533.3 → 516.3 |
| | | | | 533.3 → 498.2 |
| | | | | 533.3 → 480.2 |
| THCA | 11.7 | 515.7 | Positive | 533.3 → 462.2 |
| (Taurohyocholic Acid) | | | | 533.3 → 516.3 |
| | | | | 533.3 → 498.2 |
| | | | | 533.3 → 480.2 |
| TDCA | 23.4 | 499.7 | Positive | 517.3 → 500.2 |
| (Taurodeoxycholic Acid) | | | | 517.3 → 464.3 |
| | | | | 517.3 → 482.3 |

Quantification of regulatory T cells. Lamina propria (LP) immune cells from the small intestine were isolated using lamina propria dissociation kit (Miltenyi Biotec) following the manufacturer's instruction. All antibodies and flow cytometry reagents were obtained from Thermo Fisher. We used FITC anti-CD4 (RM4-5), PE anti-Foxp3 (FJK-16s), PE-Cyanine 7 anti-GATA3 (TWAJ), APC anti ROR gamma t (AFKJS-9), APC-efluor780 anti-Helios (22F6) and e-fluor 450 anti-CD3 (17A2) antibodies. To block non-antigen specific binding of immunoglobulins and discriminate live from dead cells, LP cell suspensions were incubated with mouse BD Fc block (BD Biosciences) and e-fluor 506 fixable viability dye (Thermo Fisher) for 30 minutes on ice. For surface staining, the cells were incubated with antibodies for 25 min, washed and fixed in fixation/permeabilization solution (Thermo Fisher). After being washed with transcription factor wash buffer (Thermo Fisher), cells were stained intracellularly for 45 min at room temperature, washed, and resuspended in PBS. Samples were read in a FACs canto analyzer (BD Biosciences) and analyzed with FACs Diva software v 8.0 (BD Biosciences). Total LP cell numbers were quantified to calculate regulatory T cell ratios. Flow Jo v10 software was used for dot plots generation and final analysis.

Statistics. Data are expressed as the mean±S.D. Statistical significance was determined by analysis of variance (ANOVA) with Tukey post hoc test, or Student's t-test, as determined by using GraphPad Prism software (version 5.0d). p values <0.05 were considered as statistically significant for in vivo animal study.

Sequencing data have been deposited in the Gene Expression Omnibus under accession number GSE104915. 16S rRNA data have been deposited to European Nucleotide Archive under accession number PRJEB26424.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, GenBank sequences, patents, and patent applications cited in this specification are herein incorporated by reference as if each was specifically and individually indicated to be incorporated by reference.

What is claimed is:

1. A method for identifying an agent that remodels gut microbiome of a subject, comprising (a) obtaining a gut microbiota sample from the subject, (b) inoculating and incubating the gut microbiota sample in a growth media in the presence of a plurality of test compounds, (c) assessing effect of the test compounds on remodeling the microbiota, and (d) identifying from the test compounds a compound that remodels the gut microbiome.

2. The method of claim 1, wherein effect of the test compounds on remodeling the microbiota is assessed by determining activity and selectivity of each test compound for remodeling the microbiota.

3. The method of claim 1, wherein the subject is afflicted with the disease that is associated with a dysfunctional gut microbiome.

4. The method of claim 3, wherein the identified compound remodels the dysfunctional gut microbiome to a functional state.

5. The method of claim 1, wherein assessing effect of the test compounds on remodeling the microbiota comprises detecting an alteration in gut microbiota transcriptome.

6. The method of claim 5, wherein alteration in gut microbiota transcriptome is detected en masse by next-generation sequencing of bacterial mRNA transcripts.

7. The method of claim 1, wherein assessing effect of the test compounds on remodeling the microbiota comprises determining the relative abundances of gut microbiota taxa by sequencing of the 16S rRNA amplicon.

8. The method of claim 6, wherein the relative bacterial abundances are used for determining the ratio of Bacteroidetes to Firmicutes.

9. The method of claim 1, wherein the identified compound remodels the microbiota sample to a desired alternative state without adversely affecting its diversity.

10. The method of claim 1, wherein the test compounds comprises a library of cyclic peptide having a sequence of from four to about sixteen amino acid residues or analogs thereof, which are alternating D- and L-residues along partial or entire sequence of the peptide.

11. A method for remodeling imbalanced or dysfunctional gut microbiota in a subject, comprising administering to the subject a pharmaceutical composition that comprises a therapeutically effective amount of a cyclic peptide, wherein the cyclic peptide comprises a sequence of from four to about sixteen amino acid residues or analogs thereof that are alternating D- and L-residues along partial or entire sequence of the peptide.

12. The method of claim 11, wherein the subject is afflicted with or at risk of developing hypercholesterolemia, a cardiovascular disorder, an atherosclerotic vascular disease, a cerebrovascular disease, aneurysm, a peripheral vascular disease or intermittent claudication.

13. The method of claim 11, wherein cyclic peptide remodels the gut microbiota in the subject to a functional state without adversely affecting its diversity.

14. The method of claim 11, wherein the pharmaceutical composition is administered to the subject orally, intravenously, subcutaneously or intraperitoneally.

15. The method of claim 11, wherein the cyclic peptide comprises alternating D- and L-α-amino acid residues along its entire sequence.

16. The method of claim 11, wherein the cyclic peptide compound has a sequence formula of c[B-J-U1-X-U2-Z],
wherein B is a peptide segment comprising at least 2 hydrophobic amino acid residues or analogs thereof; J comprises a positively charged amino acid residue, a polar uncharged amino acid residue, or an analog thereof; one or both of U1 and U2 comprise a negatively charged amino acid residue, a polar uncharged amino acid residue or analog thereof; X comprises a polar uncharged amino acid residue, a His residue or an analog thereof; and Z comprises Asn, Gln, a charged amino acid residue, or an analog thereof; and
wherein amino acid residues or analogs of the cyclic peptide are alternating D- and L-residues along the entire sequence of the cyclic peptide.

17. The method of claim 16, wherein B consists of 2, 3, 4, 5, 6, or 7 hydrophobic amino acid residues or analogs thereof.

18. The method of claim 16, wherein B consists of 3 hydrophobic amino acid residues or analogs thereof.

19. The method of claim 18, wherein B consists of ^{D}Trp-Leu-^{D}Trp, ^{D}Tyr-Leu-^{D}Tyr, ^{D}Trp-Trp-^{D}Trp, ^{D}Phe-Leu-^{D}Trp, Trp-^{D}Leu-Trp, Tyr-^{D}Leu-Tyr, Trp-^{D}Trp-Trp, or Phe-^{D}Leu-Trp.

20. The method of claim 16, wherein J is Lys, Arg, Ser, His, Orn (ornithine), diaminobutyric acid or diaminopropionic acid.

21. The method of claim 16, wherein J is naphthylalanine (Nal), homoleucine (Hml), or 2-amino-octanoic acid (Aoc).

22. The method of claim 16, wherein U1 and U2 are each independently a ^{D}Asp, ^{D}Glu or ^{D}Ser residue.

23. The method of claim 16, wherein X is Asn or Gln.

24. The method of claim 16, wherein Z is a positively charged residue.

25. The method of claim 24, wherein Z is a Lys, Arg, His, Orn (ornithine) or diaminobutyric acid.

26. The method of claim 16, wherein B consists of ^{D}Trp-Leu-^{D}Trp or ^{D}Tyr-Leu-^{D}Tyr; J is Lys, Arg, or Ser; U1 and U2 are each independently a ^{D}Asp, ^{D}Glu or ^{D}Ser residue; X is Asn or Gln; and Z is Lys, Arg, Orn (ornithine) or diaminobutyric acid.

27. The method of claim 16, wherein the cyclic peptide is selected from the group consisting of c[wLwReQeR] (SEQ ID NO:11), c[wLwKhShK] (SEQ ID NO:1), c[wLwKkKr] (SEQ ID NO:17), c[WlWlKhKr] (SEQ ID NO:18), c[wLfKwKkK] (SEQ ID NO:2), c[WlWwKkKk] (SEQ ID NO:20), c[wLhLwKrK] (SEQ ID NO:21), c[WlWlKrFr] (SEQ ID NO:19), c[FwHlYoHq] (SEQ ID NO:12), c[WlLlKkKs] (SEQ ID NO:7), c[wLlWkKkS] (SEQ ID NO:5), c[wWwKsKsK] (SEQ ID NO:8), c[lLwHoK] (SEQ ID NO:24), c[wLyKkK] (SEQ ID NO:22), c[wFkSkSkS] (SEQ ID NO:3), and c[lFlAlKhK] (SEQ ID NO:10), c[WwLlHsKk (SEQ ID NO:4), c[YlYlYkSo] (SEQ ID NO:14), c[fWwYqHhQ] (SEQ ID NO:15), c[fVwYkK] (SEQ ID NO:23), c[LlWhQk] (SEQ ID NO:6), c[WwQoHdKt] (SEQ ID NO:27), c[WlWlWkSk] (SEQ ID NO:9), c[wLeLwKsK] (SEQ ID NO:16), c[wLwSeQhK] (SEQ ID NO:25), c[YlWyKhAe] (SEQ ID NO:13), c[YwElYsKq] (SEQ ID NO:26), c[wLwSeQeO] (SEQ ID NO:28), and c[wLlEeKkN] (SEQ ID NO:29).
